(19) United States
(12) Patent Application Publication
(10) Pub. No.: US 2008/0107614 A1
(43) Pub. Date: May 8, 2008

(54) PEPTIDE-BASED CONDITIONERS

(76) Inventors: Stephen R. Fahnestock, Wilmington, DE (US); John P. O'Brien, Oxford, PA (US); Hong Wang, Kennett Square, PA (US)

Correspondence Address: E I DU PONT DE NEMOURS AND COMPANY;LEGAL PATENT RECORDS CENTER, BARLEY MILL PLAZA 25/1122B, 4417 LANCASTER PIKE, WILMINGTON, DE, 19805, US

(21) Appl. No.: 11/923,829

(22) Filed: Oct. 25, 2007

Related U.S. Application Data
(60) Provisional application No. 60/857,105, filed on Nov. 6, 2006.

(51) Int. Cl. A61K 8/64; A61Q 19/00; A61Q 3/00; A61Q 5/00; C07K 14/00; C07K 14/78; C07K 7/00

(52) U.S. Cl. 424/61; 424/70.1; 514/2; 530/324; 530/326; 530/327; 530/328; 530/329; 530/330; 530/331; 530/350

(57) ABSTRACT

Peptides have been identified that bind with high affinity to hair, skin, and nails. The peptide-based conditioners consist of a body surface-binding peptide coupled to a conditioning peptide. Conditioning peptides are typically derived from proteins and peptide having repeating amino acid sequences. Personal care compositions containing these peptide-based conditioners are also described.

This application claims the benefit of U.S. Provisional Application No. 60/857,105 filed Nov. 6, 2006.

### FIELD OF INVENTION

The invention relates to peptide-based conditioners and their use in the field of personal care products. More specifically, the invention relates to skin, hair and nail peptide-based conditioners comprising at least one body-surface binding peptide linked with at least one conditioning peptide.

### BACKGROUND OF THE INVENTION

Film-forming substances are widely used in compositions for skin and hair care as conditioning agents and moisturizers, and to protect the skin and hair against environmental and chemical damage. These substances adsorb onto and/or absorb into the skin or hair, forming a protective coating. Commonly used film-forming substances include synthetic polymers, such as silicones, polyvinylpyrrolidone, acrylic acid polymers, polysaccharides, and proteins, such as collagen, keratin, elastin, casein, silk, and soy proteins. Many proteins are known to be particularly effective film-forming agents. Because of their low solubility at the conditions used in skin and hair care products, proteins are commonly used in the form of peptides, formed by the hydrolysis of the proteins.

In hair care and hair conditioning compositions, film-forming substances are used to form a protective film on the surface of the hair to protect it from damage due to grooming and styling, shampooing, and exposure to ultraviolet light and the reactive chemicals commonly used in permanent wave agents, hair coloring products, bleaches, and hair straighteners, which denature the hair keratin protein. Moreover, these film-forming substances improve the elasticity of the hair. Film-forming substances that have been used in hair care products include proteins, such as keratin, collagen, soy, and silk and hydrolysates thereof, and polymeric materials, such as polyacrylates, long chain alkyl quaternized amines, and siloxane polymers. For example, Cannell at al. in U.S. Pat. No. 6,013,250 describe a hair care composition for treating hair against chemical and ultraviolet light damage. That composition comprises hydrolyzed protein, having an abundance of anionic amino acids, particularly, sulfur-containing amino acids, and divalent cations. It is proposed in that disclosure that the anionic components of the hydrolyzed protein bind to the hair by means of cationic bridges. Amino acids and their derivatives have also been used in hair care compositions to condition and strengthen hair. For example, O'Toole et al. in WO00/51556 describe hair care compositions containing four or more amino acid compounds selected from histidine, lysine, methionine, tyrosine, tryptophan, and cysteine compounds.

Film-forming substances are also used in skin care compositions to form a protective film on the skin. These films can serve to lubricate and coat the skin to passively impede the evaporation of moisture and smooth and soften the skin. Commonly used film-forming substances in skin care compositions include hydrolyzed animal and vegetable proteins (Puchalski et al., U.S. Pat. No. 4,416,873, El-Menshawy et al., U.S. Pat. No. 4,482,537, and Kojima et al., JP 02311412) and silk proteins (Philippe et al., U.S. Pat. No. 6,280,747 and Fahnestock et al., U.S. Pat. No. 7,060,260). Amino acids and derivatives have also been used in skin care compositions as conditioning agents. For example, Kojima et al. in JP 06065049 describe skin care compositions containing amino acids and/or their derivatives and docosahexaenoic acid, its salts or its esters. Additionally, Collier et al., U.S. Patent Publication 2004/0234609 and Kumar et al. U.S. Patent Publication 2005/0142094 use repeated sequences of amino acids to condition body surfaces; however, these molecules are not targeted to body surfaces and therefore such techniques lack lasting effectiveness.

The major problem with the current skin and hair conditioners is that they lack the durability required for long-lasting effects. For this reason, there have been attempts to enhance the binding of the cosmetic agent to the hair, or skin. For example, Richardson et al. in U.S. Pat. No. 5,490,980 and Green et al. in U.S. Pat. No. 6,267,957 describe the covalent attachment of cosmetic agents, such as skin conditioners, hair conditioners, coloring agents, sunscreens and perfumes, to hair, skin and nails using the enzyme transglutaminase. This enzyme crosslinks an amine moiety on the cosmetic agent to the glutamine residues in skin, hair and nails. Similarly, Green et al. in WO 0107009 describe the use of the enzyme lysine oxidase to covalently attach cosmetic agents to hair, skin, and nails.

In another approach, cosmetic agents have been covalently attached to proteins or protein hydrolysates. For example, Lang et al. in U.S. Pat. No. 5,192,332 describe temporary coloring compositions that contain an animal or vegetable protein, or hydrolysate thereof, which contain residues of dye molecules grafted onto the protein chain. In those compositions, the protein serves as a conditioning agent and does not enhance the binding of the cosmetic agent to hair, skin, or nails. Horikoshi et al. in JP 08104614 and Igarashi et al. in U.S. Pat. No. 5,597,386 describe hair coloring agents that consist of an anti-keratin antibody covalently attached to a dye or pigment. The antibody binds to the hair, thereby enhancing the binding of the hair coloring agent to the hair. However, neither Horikoshi et al. nor Igarashi et al. describe antibodies covalently bound to conditioning agent or as conditioning agents themselves.

Kizawa et al. in JP 09003100 describe an antibody that recognizes the surface layer of hair and its use to treat hair. A hair coloring agent consisting of that anti-hair antibody coupled to colored latex particles is also described. The use of antibodies to enhance the binding of dyes to the hair is effective in increasing the durability of the hair coloring, but these antibodies are difficult and expensive to produce. Terada et al. in JP 2002363026 describe the use of conjugates consisting of single-chain antibodies, preferably anti-keratin, coupled to dyes, ligands, and cosmetic agents for skin and hair care compositions. The single-chain antibodies may be prepared using genetic engineering techniques, but are still difficult and expensive to prepare because of their large size. Findlay in WO 00048558 describes the use of calycin proteins, such as β-lactoglobulin, which contain a binding domain for a cosmetic agent and another binding domain that binds to at least a part of the surface of a hair fiber or skin surface, for conditioners, dyes, and perfumes. Again these proteins are large and difficult and expensive to produce.

Linter in U.S. Pat. No. 6,620,419 describes peptides grafted to a fatty acid chain and their use in cosmetic and dermopharmaceutical applications. The peptides described in that disclosure are chosen because they stimulate the synthesis of collagen; they are not specific binding peptides that enhance the durability of hair and skin conditioners.

Since its introduction in 1985, phage display has been widely used to discover a variety of ligands including peptides, proteins and small molecules for drug targets (Dixit, J. of Sci. & Ind. Research, 57:173-183 (1998)). The applications have expanded to other areas such as studying protein folding, novel catalytic activities, DNA-binding proteins with novel specificities, and novel peptide-based biomaterial scaffolds for tissue engineering (Hoess, Chem. Rev. 101:3205-3218 (2001) and Holmes, Trends Biotechnol. 20:16-21 (2002)). Whaley et al. (Nature 405:665-668 (2000)) disclose the use of phage display screening to identify peptide sequences that can bind specifically to different crystallographic forms of inorganic semiconductor substrates.

A modified screening method that comprises contacting a peptide library with an anti-target to remove peptides that bind to the anti-target, then contacting the non-binding peptides with the target has been described (Estell et al. WO 0179479, Murray et al. U.S. Patent Application Publication No. 2002/0098524, and Janssen et al. U.S. Patent Application Publication No. 2003/0152976). Using that method, a peptide sequence that binds to hair and not to skin, and a peptide sequence that binds to skin and not hair, were identified. Using the same method, Janssen et al. (WO 04048399) identified other skin-binding and hair-binding peptides, as well as several binding motifs.

Although the potential use of these peptides in personal care applications is suggested in those disclosures, the covalent coupling of these peptides to conditioning agents to prepare high-affinity hair conditioners, skin conditioners and nail conditioners is not described. A method for identifying high-affinity phage-peptide clones is also described in those disclosures. The method involves using PCR to identify peptides that remain bound to the target after acid elution.

Reisch (Chem. Eng. News 80:16-21 (2002)) reports that a family of peptides designed to target an ingredient of specific human tissue has been developed for personal care applications. However, no description of peptide-based conditioners are disclosed in that publication.

In view of the above, a need exists for conditioners that may be applied to body surfaces such as hair, skin and nails that provide improved durability for long lasting effects and are easy and inexpensive to prepare.

Applicants have met the stated need by creating peptide conjugates comprising peptides that have a binding affinity for body surfaces such as hair, skin and nails, functionally linked to a conditioning peptide derived from various repetitively sequenced proteins, such as silk.

### SUMMARY OF THE INVENTION

The invention provides peptide conjugates comprising body surface-binding peptides linked to a conditioning peptide that is derived from a repetitively sequenced peptide. The two portions of the conjugate may be contiguous or separated by a spacer. The conjugates of the invention are useful in personal care conditioning reagents for conditioning hair, skin and nails.

Accordingly the invention provides A peptide based conditioning reagent having the general structure [[(BSBP)ₘ-S_{q}]ₓ—[(CP)ₙ—Sᵣ]_{z}]_{y}, wherein
a) BSBP is a body surface-binding peptide;
b) CP is a conditioning peptide;
c) S is a molecular spacer; and
d) m, n, x and z independently range from 1 to about 10, y is from 1 to about 5, and where q and r are each independently 0 or 1, and wherein the peptide based conditioning reagent has a molecular weight of less than about 200,000 Daltons.

In an alternate embodiment the body surface-binding peptide of the invention may be produced by a process comprising the steps of:
(i) providing a library of combinatorially generated phage-peptides;
(ii) contacting the library of (i) with a body surface to form a reaction solution comprising:
   (A) phage-peptide-body surface complex;
   (B) unbound body surface, and
   (C) uncomplexed peptides;
(iii) isolating the phage-peptide-body surface complex of (ii);
(iv) eluting the weakly bound peptides from the isolated peptide complex of (iii);
(v) identifying the remaining bound phage-peptides either by using the polymerase chain reaction directly with the phage-peptide-body surface complex remaining after step (iv), or by infecting bacterial host cells directly with the phage-peptide-body surface complex remaining after step (iv), growing the infected cells in a suitable growth medium, and isolating and identifying the phage-peptides from the grown cells.

In another embodiment the invention provides a personal care composition comprising an effective amount of the peptide-based conditioning reagent of the invention, comprising a body surface-binding peptide and a conditioning peptide.

In an alternate embodiment the invention provides a method for conditioning a body surface comprising applying a personal care composition comprising an effective amount of the peptide-based conditioning reagent as described above, comprising a body surface-binding peptide and a conditioning peptide, to a body surface under conditions wherein the body surface is conditioned.

### BRIEF DESCRIPTION OF FIGURES AND SEQUENCE DESCRIPTIONS

FIG. 1 is a plasmid map of the vector pKSIC4-HC77623, described in Example 10.

The invention can be more fully understood from the following detailed description and the accompanying sequence descriptions, which form a part of this application.

The following sequences conform with 37 C.F.R. 1.821-1.825 (“Requirements for patent applications Containing Nucleotide Sequences and/or Amino Acid Sequence Disclosures—the Sequence Rules”) and are consistent with World Intellectual Property Organization (WIPO) Standard ST.25 (1998) and the sequence listing requirements of the EPO and PCT (Rules 5.2 and 49.5(a-bis), and Section 208 and Annex C of the Administrative Instructions). The symbols and format used for nucleotide and amino acid sequence data comply with the rules set forth in 37 C.F.R. §1.822.

The following Table A identifies the sequences referenced in the present application:

TABLE A

| | Amino Acid/ | |
|---|---|---|
| SEQ ID NO | Nucleic acid | Sequence Description |
| 1 | Amino Acid | Hair-Binding peptide |
| 2 | Amino Acid | Skin-Binding peptide |
| 3 | Amino Acid | Hair-binding peptide |
| 4 | Amino Acid | Hair-binding peptide |
| 5 | Amino Acid | Hair-binding peptide |
| 6 | Amino Acid | Hair-binding peptide |
| 7 | Amino Acid | Hair-binding peptide |
| 8 | Amino Acid | Hair-binding peptide |
| 9 | Amino Acid | Hair-binding peptide |
| 10 | Amino Acid | Hair-binding peptide |
| 11 | Amino Acid | Hair-binding peptide |
| 12 | Amino Acid | Hair-binding peptide |
| 13 | Amino Acid | Hair-binding peptide |
| 14 | Amino Acid | Hair-binding peptide |
| 15 | Amino Acid | Hair-binding peptide |
| 16 | Amino Acid | Hair-binding peptide |
| 17 | Amino Acid | Hair-binding peptide |
| 18 | Amino Acid | Hair-binding peptide |
| 19 | Amino Acid | Hair-binding peptide |
| 20 | Amino Acid | Hair-binding peptide |
| 21 | Amino Acid | Hair-binding peptide |
| 22 | Amino Acid | Hair-binding peptide |
| 23 | Amino Acid | Hair-binding peptide |
| 24 | Amino Acid | Hair-binding peptide |
| 25 | Amino Acid | Hair-binding peptide |
| 26 | Amino Acid | Hair-binding peptide |
| 27 | Amino Acid | Hair-binding peptide |
| 28 | Amino Acid | Hair-binding peptide |
| 29 | Amino Acid | Hair-binding peptide |
| 30 | Amino Acid | Hair-binding peptide |
| 31 | Amino Acid | Hair-binding peptide |
| 32 | Amino Acid | Hair-binding peptide |
| 33 | Amino Acid | Hair-binding peptide |
| 34 | Amino Acid | Hair-binding peptide |
| 35 | Amino Acid | Hair-binding peptide |
| 36 | Amino Acid | Hair-binding peptide |
| 37 | Amino Acid | Hair-binding peptide |
| 38 | Amino Acid | Hair-binding peptide |
| 39 | Amino Acid | Hair-binding peptide |
| 40 | Amino Acid | Hair-binding peptide |
| 41 | Amino Acid | Hair-binding peptide |
| 42 | Amino Acid | Hair-binding peptide |
| 43 | Amino Acid | Hair-binding peptide |
| 44 | Amino Acid | Hair-binding peptide |
| 45 | Amino Acid | Hair-binding peptide |
| 46 | Amino Acid | Hair-binding peptide |
| 47 | Amino Acid | Hair-binding peptide |
| 48 | Amino Acid | Hair-binding peptide |
| 49 | Amino Acid | Hair-binding peptide |
| 50 | Amino Acid | Hair-binding peptide |
| 51 | Amino Acid | Hair-binding peptide |
| 52 | Amino Acid | Hair-binding peptide |
| 53 | Amino Acid | Hair and Nail-binding peptide |
| 54 | Amino Acid | Hair-binding peptide |
| 55 | Amino Acid | Hair-binding peptide |
| 56 | Amino Acid | Hair-binding peptide |
| 57 | Amino Acid | Hair-binding peptide |
| 58 | Amino Acid | Hair-binding peptide |
| 59 | Amino Acid | Hair-binding peptide |
| 60 | Amino Acid | Nail-binding peptide |
| 61 | Amino Acid | Skin-binding peptide |
| 62 | Nucleic Acid | Sequencing primer |
| 63 | Amino Acid | Control Peptide |
| 64 | Amino Acid | Hair-binding peptide with C-terminal |
| | | cysteine addition |
| 65 | Amino Acid | Amino acid sequence of Caspase 3 |
| | | cleavage site sequence |
| 66 | Amino Acid | Shampoo resistant hair-binding |
| | | peptide |
| 67 | Nucleic acid | Primer |
| 68 | Nucleic acid | Primer |
| 69 | Amino Acid | Shampoo resistant hair-binding |
| | | peptide |
| 70 | Amino Acid | Shampoo resist hair-binding peptide |
| 71 | Amino Acid | Biotinylated hair-binding peptide |
| 72 | Amino Acid | Biotinylated hair/skin-binding peptide |
| 73 | Amino Acid | Biotinylated hair-binding peptide |
| 74 | Amino Acid | Biotinylated skin-binding peptide |
| 75 | Amino Acid | Hair-binding peptide |
| 76 | Amino Acid | Hair-binding peptide |
| 77 | Amino Acid | Hair-binding peptide |
| 78 | Amino Acid | Hair-binding peptide |
| 79 | Amino Acid | Hair-binding peptide |
| 80 | Amino Acid | Hair-binding peptide |
| 81 | Amino Acid | Hair-binding peptide |
| 82 | Amino Acid | Hair-binding peptide |
| 83 | Amino Acid | Hair-binding peptide |
| 84 | Amino Acid | Hair-binding peptide |
| 85 | Amino Acid | Hair-binding peptide |
| 86 | Amino Acid | Hair-binding peptide |
| 87 | Amino Acid | Hair-binding peptide |
| 88 | Amino Acid | Hair-binding peptide |
| 89 | Amino Acid | Hair-binding peptide |
| 90 | Amino Acid | Hair-binding peptide |
| 91 | Amino Acid | Hair-binding peptide |
| 92 | Amino Acid | Hair-binding peptide |
| 93 | Amino Acid | Hair-binding peptide |
| 94 | Amino Acid | Hair-binding peptide |
| 95 | Amino Acid | Hair-binding peptide |
| 96 | Amino Acid | Hair-binding peptide |
| 97 | Amino Acid | Hair-binding peptide |
| 98 | Amino Acid | Skin-binding peptide |
| 99 | Amino Acid | Skin-binding peptide |
| 100 | Amino Acid | Skin-binding peptide |
| 101 | Amino Acid | Skin-binding peptide |
| 102 | Amino Acid | Skin-binding peptide |
| 103 | Amino Acid | Skin-binding peptide |
| 104 | Amino Acid | Empirically generated Hair and Skin- |
| | | binding peptide |
| 105 | Amino Acid | Empirically generated Hair and Skin- |
| | | binding peptide |
| 106 | Amino Acid | Empirically generated Hair and Skin- |
| | | binding peptide |
| 107 | Amino Acid | Empirically generated Hair and Skin- |
| | | binding peptide |
| 108 | Amino Acid | Empirically generated Hair and Skin- |
| | | binding peptide |
| 109 | Amino Acid | Peptide spacer |
| 110 | Amino Acid | Peptide spacer |
| 111 | Amino Acid | Peptide spacer |
| 112 | Amino Acid | Conditioner and Shampoo Resistant |
| | | Hair-binding peptide |
| 113 | Amino Acid | Conditioner and Shampoo Resistant |
| | | Hair-binding peptide |
| 114 | Amino Acid | Conditioner and Shampoo Resistant |
| | | Hair-binding peptide |
| 115 | Amino Acid | Conditioner and Shampoo Resistant |
| | | Hair-binding peptide |
| 116 | Amino Acid | Hair-binding peptide |
| 117 | Amino Acid | Conditioning peptide |
| 118 | Amino Acid | Conditioning peptide |
| 119 | Amino Acid | Conditioning peptide |
| 120 | Amino Acid | Conditioning peptide |
| 121 | Amino Acid | Conditioning peptide |
| 122 | Amino Acid | Conditioning peptide |
| 123 | Amino Acid | Peptide spacer |
| 124 | Amino Acid | Peptide spacer |
| 125 | Amino Acid | Hair-binding peptide |
| 126 | Amino Acid | Conditioning peptide -Silk |
| 127 | Amino Acid | Conditioning peptide -Elastin |
| 128 | Amino Acid | Conditioning peptide - Abductin |
| 129 | Amino Acid | Conditioning peptide - Byssus |
| 130 | Amino Acid | Conditioning peptide - Gluten |
| 131 | Amino Acid | Conditioning peptide -Gluten |
| 132 | Amino Acid | Conditioning peptide - Titin |
| 133 | Amino Acid | Conditioning peptide - Extensin |
| 134 | Amino Acid | Conditioning peptide - Fibronectin |
| 135 | Amino Acid | Conditioning peptide - Gliaden |
| 136 | Amino Acid | Conditioning peptide - Glue |
| 137 | Amino Acid | Conditioning peptide - Nucleating |
| 138 | Amino Acid | Conditioning peptide - Keratin |
| 139 | Amino Acid | Conditioning peptide - Keratin |
| 140 | Amino Acid | Conditioning peptide - Mucin |
| 141 | Amino Acid | Conditioning peptide - RNA |
| | | Polymerase |
| 142 | Amino Acid | Conditioning peptide - Silk fibroin- |
| | | like |
| 143 | Amino Acid | Conditioning peptide - Silk A repeat |
| 144 | Amino Acid | Conditioning peptide - Silk E repeat |
| 145 | Amino Acid | Conditioning peptide -Silk S repeat |
| 146 | Amino Acid | Conditioning peptide -Silk |
| | | consensus |
| 147 | Amino Acid | Conditioning peptide -spider |
| | | dragline silk |
| 148 | Amino Acid | Conditioning peptide -spideroid |
| | | DP1A |
| 149 | Amino Acid | Conditioning p Conditioning peptide |
| | | -spideroid DP1B |
| 150 | Amino Acid | Conditioning peptide -spider |
| | | dragline silk |
| 151 | Amino Acid | Conditioning peptide -spider |
| | | dragline silk |
| 152 | Amino Acid | Conditioning peptide -spider |
| | | dragline silk |
| 153 | Amino Acid | Conditioning peptide -spider |
| | | dragline silk |
| 154 | Amino Acid | Conditioning peptide -spider |
| | | dragline silk |
| 155 | Amino Acid | Conditioning peptide -spider |
| | | dragline silk |
| 156 | Amino Acid | Conditioning peptide -spider |
| | | dragline silk |
| 157 | Amino Acid | Conditioning peptide -spider |
| | | dragline silk |
| 158 | Amino Acid | Conditioning peptide - silk like |
| 159 | Amino Acid | Peptide spacer |
| 160 | Amino Acid | Conditioning peptide - silk like |
| 161 | Amino Acid | Peptide conjugate HC77648 |
| 162 | Amino Acid | Conditioning peptide - Keratinx4 |
| 163 | Amino Acid | Peptide conjugate - HC77649 |
| 164 | Amino Acid | Conditioning peptide - Keratinx3 |
| 165 | Amino Acid | Conditioning peptide - Beta Silkx4 |
| 166 | Amino Acid | Peptide conjugate HC77651 |
| 167 | Nucleic Acid | Nucleic acid sequence encoding |
| | | peptide conjugate HC77648 |
| 168 | Nucleic Acid | Nucleic acid sequence encoding |
| | | peptide conjugate HC77649 |
| 169 | Nucleic Acid | Nucleic acid sequence encoding |
| | | peptide conjugate HC77651 |
| 170 | Amino Acid | Conditioning peptide - gluten-like |
| 171 | Nucleic Acid | PCR primer -96 gIII |
| 172 | Nucleic Acid | Expression Plasmid pKSIC4- |
| | | HC77623 |
| 173 | Amino Acid | Conditioning peptide - silk-like |
| 174 | Amino Acid | Conditioning peptide - silk fibroin- |
| | | like repeat sequence |
| 175 | Amino Acid | Conditioning peptide - silk and |
| | | elastin-like repeat sequence |
| 176 | Amino Acid | Conditioning peptide - repeat |
| | | sequence |
| 177 | Amino Acid | Conditioning peptide - repeat |
| | | sequence |
| 178 | Amino Acid | Conditioning peptide - synthetic |
| | | glycine rich repeat sequence |
| 179 | Amino Acid | Conditioning peptide - |
| | | metallothionin like peptide segments |
| 180 | Amino Acid | Conditioning peptide - synthetic |
| | | glycine rich repeat sequences |
| 181 | Amino Acid | Conditioning peptide - synthetic |
| | | glycine rich repeat sequences |
| 182 | Amino Acid | Conditioning peptide - silk and |
| | | elastin-like repeat sequences |
| 183 | Amino Acid | Conditioning peptide - silk and |
| | | elastin repeat sequences |
| 184 | Amino Acid | Conditioning peptide - silk and |
| | | elastin-like repeat sequences |
| 185 | Amino Acid | Conditioning peptide - silk and |
| | | elastin-like repeat sequences |
| 186 | Amino Acid | Conditioning peptide - synthetic |
| | | repeat sequences |
| 187 | Amino Acid | Conditioning peptide - silk and |
| | | elastin-like repeat sequences |
| 188 | Amino Acid | Conditioning peptide - silk and |
| | | elastin-like repeat sequences |
| 189 | Amino Acid | Conditioning peptide - silk, elastin, |
| | | and MBI repeat sequences |
| 190 | Amino Acid | Conditioning peptide - GFP-SELPK |
| | | silk, elastin, and green fluorescent |
| | | protein peptides |
| 191 | Amino Acid | Conditioning peptide |
| 192 | Amino Acid | Conditioning peptide - P-SELPK, |
| | | elastin, and UV-protective peptide |
| | | sequences |
| 193 | Amino Acid | Conditioning peptide - CBFxamer- |
| | | SELPK silk, elastin, and cellulose- |
| | | binding peptide polymer sequence |
| 194 | Amino Acid | Conditioning peptide - SELP 47R-3 |
| 195 | Amino Acid | Conditioning peptide - SELP 67K |
| 196 | Amino Acid | Conditioning peptide - SELP47K-P4 |
| 197 | Amino Acid | Conditioning peptide - DCP6 |

### DETAILED DESCRIPTION OF THE INVENTION

The present invention provides peptide sequences that specifically bind to human hair, skin, nails and substitutes thereof with high affinity. Additionally, the present invention provides peptide-based hair, skin and nail conditioners with improved durability. The binding peptides coupled to the conditioning peptides of the invention are useful as hair, skin and nail conditioning agents.

The following definitions are used herein and should be referred to for interpretation of the claims and the specification.

The term “invention” or “present invention” as used herein is a non-limiting term and is not intended to refer to a single embodiment of the particular invention but encompasses all possible embodiments as described in the specification and the claims.

“BSBP” as used herein means body surface-binding peptide.

“HBP” as used herein means hair-binding peptide.

“SBP” as used herein means skin-binding peptide.

“NBP” as used herein means nail-binding peptide.

“BP” as used herein means binding peptide of either skin-, nail- or hair-binding type.

“CP” as used herein means conditioning peptide. “Conditioning peptide” means any peptide that improves the quality of a body surface. A conditioning peptide will be one that is derived from a repetitively sequenced peptide and will have film forming properties.

The term “peptide conjugate” refers to the conjugate of a body surface-binding peptide with a conditioning peptide. Within the conjugate the two peptide portions or domains may be separated by a peptide or molecular spacer. As such the peptides of the conjugate are said to be “functionally linked”, meaning that each peptide is associated with the other peptides in a manner that allows that peptide to perform its respective function.

“Repeat sequence protein” refers to proteins comprising multiple repeats of a series of amino acids derived from natural structure supporting materials such as silk, elastin, collagen, dragline silk, fibronectin, keratin and the like.

The term “silk-like protein” will be abbreviated “SLP” and refers to natural silk proteins and their synthetic analogs having the following three criteria: (1) amino acid composition of the molecule is dominated by glycine and/or alanine; (2) consensus crystalline domain is arrayed repeatedly throughout the molecule; (3) the molecule is shear sensitive and can be spun into semicrystalline fiber. SLP's should also include molecules which are the modified variants of the natural silk proteins and their synthetic analogs defined above.

The terms “peptide”, “polypeptide” and “protein” are used interchangeably and refer to two or more amino acids joined to each other by peptide bonds or modified peptide bonds.

The term “spider silk variant protein” will refer to a designed protein, the amino acid sequence of which is based on repetitive sequence motifs and variations thereof that are found in a known natural spider silk.

The term “DP-1B” will refer to any spider silk variant derived from the amino acid sequence of the natural Protein 1 (Spidroin 1) of Nephila calvipes as set forth in SEQ ID NO:149.

“S” as used herein means spacer. “Spacer” or “linker” will be used interchangeably and will refer to an entity that links the body surface-binding peptide with the conditioning peptide. The spacer or linker may be comprised of amino acids or may be a chemical linker.

The term “body surface” refers to any surface of the human body that may serve as a substrate for the binding of a diblock or triblock peptide-based body surface conditioning reagent comprising at least one body surface-binding peptide and at least one conditioning peptide. Typical body surfaces include, but are not limited to hair, skin, and nails.

The term “hair” as used herein refers to human hair, eyebrows, and eyelashes.

The term “skin” as used herein refers to human skin, or substitutes for human skin especially pig skin, VITRO-SKIN® and EPIDERM®.

The term “nails” as used herein refers to human fingernails and toenails.

The term “stringency” as it is applied to the selection of the hair-binding and skin-binding of the present invention, refers to the concentration of the eluting agent (usually detergent) used to elute peptides from the hair or skin. Higher concentrations of the eluting agent provide more stringent conditions.

The term “peptide-hair complex” as used herein means structure comprising a peptide or polypeptide bound to a hair fiber via a binding site on the peptide.

The term “peptide-skin complex” as used herein means structure comprising a peptide or polypeptide bound to the skin via a binding site on the peptide.

The term “peptide-nail complex” as used herein means structure comprising a peptide or polypeptide bound to nails via a binding site on the peptide.

The term “peptide-substrate complex” refers to either peptide-hair, peptide-skin, or peptide-nail complexes.

The term “phage-peptide-body surface complex” as used herein means structure comprising a phage-displayed peptide or polypeptide bound to a body surface.

The term “functional group” as used herein means a region of a peptide or polypeptide designed, suspected, or known, to have a specific function or a chemical unit bound to a peptide that provides the complex with a specific function. As used herein either terminal end of a peptide can be considered a functional group as that region is specific in function. Non-limiting examples of other functional groups include body surface-binding peptides, conditioning peptides, and spacers.

The term “diblock” as used herein means a complex formed of two types of primary functional groups. Each functional group may be represented by one or many members. Other minor functional groups beyond the primary two may be present in a diblock.

The term “triblock” as used herein means a complex formed of three types of primary functional groups. Each functional group may be represented by one or many members. Other minor functional groups beyond the primary three may be present in a triblock.

The term “MB₅₀” refers to the concentration of the binding peptide that gives a signal that is 50% of the maximum signal obtained in an ELISA-based binding assay as described herein. The MB₅₀ provides an indication of the strength of the binding interaction or affinity of the components of the complex. The lower the value of MB₅₀, the stronger the interaction of the peptide with its corresponding substrate.

The term “binding affinity” refers to the strength of the interaction of a binding peptide with its respective substrate. The binding affinity is defined herein in terms of the MB₅₀ value, determined in an ELISA-based binding assay.

The term “amino acid” refers to the basic chemical structural unit of a protein or polypeptide. The following abbreviations are used herein to identify specific amino acids:

| | Three-Letter | One-Letter |
|---|---|---|
| Amino Acid | Abbreviation | Abbreviation |
| Alanine | Ala | A |
| Arginine | Arg | R |
| Asparagine | Asn | N |
| Aspartic acid | Asp | D |
| Cysteine | Cys | C |
| Glutamine | Gln | Q |
| Glutamic acid | Glu | E |
| Glycine | Gly | G |
| Histidine | His | H |
| Isoleucine | Ile | I |
| Leucine | Leu | L |
| Lysine | Lys | K |
| Methionine | Met | M |
| Phenylalanine | Phe | F |
| Proline | Pro | P |
| Serine | Ser | S |
| Threonine | Thr | T |
| Tryptophan | Trp | W |
| Tyrosine | Tyr | Y |
| Valine | Val | V |
| Any (or as defined herein) | Xaa | X |

“Gene” refers to a nucleic acid fragment that expresses a specific peptide, polypeptide or protein, including regulatory sequences preceding (5′ non-coding sequences) and following (3′ non-coding sequences) the coding sequence. “Native gene” refers to a gene as found in nature with its own regulatory sequences “Chimeric gene” refers to any gene that is not a native gene, comprising regulatory and coding sequences that are not found together in nature. Accordingly, a chimeric gene may comprise regulatory sequences and coding sequences that are derived from different sources, or regulatory sequences and coding sequences derived from the same source, but arranged in a manner different than that found in nature. A “foreign” gene refers to a gene not normally found in the host organism, but that is introduced into the host organism by gene transfer. Foreign genes can comprise native genes inserted into a non-native organism, synthetic gene, or chimeric genes.

“Synthetic genes” can be assembled from oligonucleotide building blocks that are chemically synthesized using procedures known to those skilled in the art. These building blocks are ligated and annealed to form gene segments which are then enzymatically assembled to construct the entire gene. “Chemically synthesized”, as related to a sequence of DNA, means that the component nucleotides were assembled in vitro. Manual chemical synthesis of DNA may be accomplished using well-established procedures, or automated chemical synthesis can be performed using one of a number of commercially available machines. Accordingly, the genes can be tailored for optimal gene expression based on optimization of nucleotide sequence to reflect the codon bias of the host cell. The skilled artisan appreciates the likelihood of successful gene expression if codon usage is biased towards those codons favored by the host. Determination of preferred codons can be based on a survey of genes derived from the host cell where sequence information is available.

“Coding sequence” refers to a DNA sequence that codes for a specific amino acid sequence. “Suitable regulatory sequences” refer to nucleotide sequences located upstream (5′ non-coding sequences), within, or downstream (3′ non-coding sequences) of a coding sequence, and which influence the transcription, RNA processing or stability, or translation of the associated coding sequence. Regulatory sequences may include promoters, translation leader sequences, introns, polyadenylation recognition sequences, RNA processing site, effector binding site and stem-loop structure.

“Promoter” refers to a DNA sequence capable of controlling the expression of a coding sequence or functional RNA. In general, a coding sequence is located 3′ to a promoter sequence. Promoters may be derived in their entirety from a native gene, or be composed of different elements derived from different promoters found in nature, or even comprise synthetic DNA segments. It is understood by those skilled in the art that different promoters may direct the expression of a gene in different tissues or cell types, or at different stages of development, or in response to different environmental or physiological conditions. Promoters which cause a gene to be expressed in most cell types at most times are commonly referred to as “constitutive promoters”. It is further recognized that since in most cases the exact boundaries of regulatory sequences have not been completely defined, DNA fragments of different lengths may have identical promoter activity.

The term “expression”, as used herein, refers to the transcription and stable accumulation of sense (mRNA) or antisense RNA derived from the nucleic acid fragment of the invention. Expression may also refer to translation of mRNA into a polypeptide.

The term “transformation” refers to the transfer of a nucleic acid fragment into the genome of a host organism, resulting in genetically stable inheritance. Host organisms containing the transformed nucleic acid fragments are referred to as “transgenic” or “recombinant” or “transformed” organisms.

The term “host cell” refers to cell which has been transformed or transfected, or is capable of transformation or transfection by an exogenous polynucleotide sequence.

The terms “plasmid”, “vector” and “cassette” refer to an extra chromosomal element often carrying genes which are not part of the central metabolism of the cell, and usually in the form of circular double-stranded DNA molecules. Such elements may be autonomously replicating sequences, genome integrating sequences, phage or nucleotide sequences, linear or circular, of a single- or double-stranded DNA or RNA, derived from any source, in which a number of nucleotide sequences have been joined or recombined into a unique construction which is capable of introducing a promoter fragment and DNA sequence for a selected gene product along with appropriate 3′ untranslated sequence into a cell. “Transformation cassette” refers to a specific vector containing a foreign gene and having elements in addition to the foreign gene that facilitate transformation of a particular host cell. “Expression cassette” refers to a specific vector containing a foreign gene and having elements in addition to the foreign gene that allow for enhanced expression of that gene in a foreign host.

The term “phage” or “bacteriophage” refers to a virus that infects bacteria. Altered forms may be used for the purpose of the present invention. The preferred bacteriophage is derived from the “wild” phage, called M13. The M13 system can grow inside a bacterium, so that it does not destroy the cell it infects but causes it to make new phage continuously. It is a single-stranded DNA phage.

The term “phage display” refers to the display of functional foreign peptides or small proteins on the surface of bacteriophage or phagemid particles. Genetically engineered phage may be used to present peptides as segments of their native surface proteins. Peptide libraries may be produced by populations of phage with different gene sequences.

“PCR” or “polymerase chain reaction” is a technique used for the amplification of specific DNA segments (U.S. Pat. Nos. 4,683,195 and 4,800,159).

Standard recombinant DNA and molecular cloning techniques used herein are well known in the art and are described by Sambrook, J. and Russell, D., Molecular Cloning: A Laboratory Manual, Third Edition, Cold Spring Harbor Laboratory Press, Cold Spring Harbor, N.Y. (2001); and by Silhavy, T. J., Bennan, M. L. and Enquist, L. W., Experiments with Gene Fusions, Cold Spring Harbor Laboratory Cold Press Spring Harbor, N.Y. (1984); and by Ausubel, F. M. et. al., Short Protocols in Molecular Biology, 5^{th} Ed. Current Protocols and John Wiley and Sons, Inc., N.Y., 2002.

The present invention comprises specific hair-binding, skin-binding, and nail-binding peptides and their use in conditioners for the hair, skin, and nails. The invention provides a conditioning compound comprised of a binding peptide that has affinity for a body surface, functionally linked to a conditioning peptide. The binding and conditioning peptides may be associated through a spacer or chemical linker and one or more of the peptides may be variously repeated.

Body Surfaces

Body surfaces of the invention are any surface on the human body that will serve as a substrate for a binding peptide. Typical body surfaces include, but are not limited to hair, skin, and nails.

Samples of body surfaces are available from a variety of sources. For example, human hair samples are available commercially, for example from International Hair Importers and Products (Bellerose, N.Y.), in different colors, such as brown, black, red, and blond, and in various types, such as African-American, Caucasian, and Asian. Additionally, the hair samples may be treated for example using hydrogen peroxide to obtain bleached hair. Human skin samples may be obtained from cadavers or in vitro human skin cultures. Additionally, pig skin, available from butcher shops and supermarkets, VITRO-SKIN®, available from IMS Inc. (Milford, Conn.), and EPIDERM®, available from MatTek Corp. (Ashland, Mass.), are good substitutes for human skin. Human fingernails and toenails may be obtained from volunteers.

Body Surface-Binding Peptides

Body surface-binding peptides as defined herein are peptide sequences that specifically bind with high affinity to specific body surfaces, including, but not limited to hair, nails, teeth, gums, skin and the tissues of the oral cavity, for example. Suitable body surface-binding peptide sequences may be selected using combinatorial methods that are well known in the art or may be empirically generated. The body surface-binding peptides of the invention have a binding affinity for their respective substrate, as measured by MB₅₀ values, of less than or equal to about 10⁻² M, less than or equal to about 10⁻³ M, less than or equal to about 10⁻⁴ M, less than or equal to about 10⁻⁵ M, preferably less than or equal to about 10⁻⁶ M, and more preferably less than or equal to about 10⁻⁷ M.

Hair-binding peptides (HBPs), skin-binding peptides (SBPs) and nail-binding peptides (NBPs) as defined herein are peptide sequences that specifically bind with high affinity to hair, skin, and nails respectively. Combinatorially generated body surface-binding peptides of the present invention are typically from about 7 amino acids to about 50 amino acids, more preferably, from about 7 amino acids to about 25 amino acids, most preferably from about 7 to about 20 amino acids.

Suitable body surface-binding sequences may be selected using methods that are well known in the art. The peptides of the present invention are generated randomly and then selected against a specific hair, skin or nail sample based upon their binding affinity for the substrate of interest. The generation of random libraries of peptides is well known and may be accomplished by a variety of techniques including, bacterial display (Kemp, D. J.; Proc. Natl. Acad. Sci. USA 78(7): 4520-4524 (1981); yeast display (Chien et al., Proc Natl Acad Sci USA 88(21): 9578-82 (1991)), combinatorial solid phase peptide synthesis (U.S. Pat. No. 5,449,754; U.S. Pat. No. 5,480,971; U.S. Pat. No. 5,585,275 and U.S. Pat. No. 5,639,603), phage display technology (U.S. Pat. No. 5,223,409; U.S. Pat. No. 5,403,484; U.S. Pat. No. 5,571,698; and U.S. Pat. No. 5,837,500), ribosome display (U.S. Pat. No. 5,643,768; U.S. Pat. No. 5,658,754; and U.S. Pat. No. 7,074,557), and mRNA display technology (PROFUSION™; U.S. Pat. No. 6,258,558; U.S. Pat. No. 6,518,018; U.S. Pat. No. 6,281,344; U.S. Pat. No. 6,214,553; U.S. Pat. No. 6,261,804; U.S. Pat. No. 6,207,446; U.S. Pat. No. 6,846,655; U.S. Pat. No. 6,312,927; U.S. Pat. No. 6,602,685; U.S. Pat. No. 6,416,950; U.S. Pat. No. 6,429,300; U.S. Pat. No. 7,078,197; and U.S. Pat. No. 6,436,665). Exemplary methods used to generate such biological peptide libraries are described in Dani, M., J. of Receptor & Signal Transduction Res., 21(4):447-468 (2001), Sidhu et al., Methods in Enzymology 328:333-363 (2000), and Phage Display of Peptides and Proteins, A Laboratory Manual, Brian K. Kay, Jill Winter, and John McCafferty, eds.; Academic Press, NY, 1996. Additionally, phage display libraries are available commercially from companies such as New England Biolabs (Beverly, Mass.).

A preferred method to randomly generate peptides is by phage display. Phage display is an in vitro selection technique in which a peptide or protein is genetically fused to a coat protein of a bacteriophage, resulting in display of fused peptide on the exterior of the phage virion, while the DNA encoding the fusion resides within the virion. This physical linkage between the displayed peptide and the DNA encoding it allows screening of vast numbers of variants of peptides, each linked to a corresponding DNA sequence, by a simple in vitro selection procedure called “biopanning”. In its simplest form, biopanning is carried out by incubating the pool of phage-displayed variants with a target of interest that has been immobilized on a plate or bead, washing away unbound phage, and eluting specifically bound phage by disrupting the binding interactions between the phage and the target. The eluted phage is then amplified in vivo and the process is repeated, resulting in a stepwise enrichment of the phage pool in favor of the tightest binding sequences. After 3 or more rounds of selection/amplification, individual clones are characterized by DNA sequencing.

After a suitable library of peptides has been generated, they are then contacted with an appropriate amount of the test substrate, specifically a hair, skin, or nail sample. The test substrate is presented to the library of peptides while suspended in solution. A preferred solution is a buffered aqueous saline solution containing a surfactant. A suitable solution is Tris-buffered saline (TBS) with 0.5% TWEEN® 20. The solution may additionally be agitated by any means in order to increase the mass transfer rate of the peptides to the hair, skin, or nail surface, thereby shortening the time required to attain maximum binding.

Upon contact, a number of the randomly generated peptides will bind to the hair, skin, or nail substrate to form a peptide-hair, peptide-skin or peptide-nail complex. Unbound peptide may be removed by washing. After all unbound material is removed, peptides having varying degrees of binding affinities for the test substrate may be fractionated by selected washings in buffers having varying stringencies. Increasing the stringency of the buffer used increases the required strength of the bond between the peptide and substrate in the peptide-substrate complex.

A number of substances may be used to vary the stringency of the buffer solution in peptide selection including, but not limited to, acidic pH (1.5-3.0); basic pH (10-12.5); high salt concentrations such as MgCl₂ (3-5 M) and LiCl (5-10 M); water; ethylene glycol (25-50%); dioxane (5-20%); thiocyanate (1-5 M); guanidine (2-5 M); urea (2-8 M); and various concentrations of different surfactants such as SDS (sodium dodecyl sulfate), DOC (sodium deoxycholate), Nonidet P-40, Triton X-100, TWEEN® 20, wherein TWEEN® 20 is preferred. These substances may be prepared in buffer solutions including, but not limited to, Tris-HCl, Tris-buffered saline, Tris-borate, Tris-acetic acid, triethylamine, phosphate buffer, and glycine-HCl, wherein Tris-buffered saline solution is preferred.

It will be appreciated that peptides having increasing binding affinities for hair, skin or nail substrates may be eluted by repeating the selection process using buffers with increasing stringencies. The eluted peptides can be identified and sequenced by any means known in the art.

Thus, the following method for generating the body surface-binding peptides of the present invention can be used. A library of combinatorially generated phage-peptides is contacted with the substrate of interest, specifically, a hair, skin or nail sample, to form a phage-peptide-body surface [phage-peptide-hair, phage-peptide-skin, or phage-peptide-nail] complexes. The phage-peptide-body surface complex is separated from uncomplexed peptides and unbound substrate, and the bound phage-peptides from the phage-peptide-body surface complexes are eluted from the complex, preferably by acid treatment. Then, the eluted peptides are identified and sequenced. To identify peptide sequences that bind to one substrate but not to another, for example peptides that bind to hair, but not to skin or peptides that bind to skin, but not to hair, a subtractive panning step is added. Specifically, the library of combinatorially generated phage-peptides is first contacted with the non-target to remove phage-peptides that bind to it. Then, the non-binding phage-peptides are contacted with the desired substrate and the above process is followed. Alternatively, the library of combinatorially generated phage-peptides may be contacted with the non-target and the desired substrate simultaneously. Then, the phage-peptide-substrate complexes are separated from the phage-peptide-non-target complexes and the method described above is followed for the desired phage-peptide-substrate complexes.

One embodiment of the present invention provides a modified phage display screening method for isolating peptides with a higher affinity for hair, skin or nails. In the modified method, the phage-peptide-substrate complexes are formed as described above. Then, these complexes are treated with an elution buffer. Any of the elution buffers described above may be used. Preferably, the elution buffer is an acidic solution. The remaining, elution-resistant phage-peptide-substrate complexes are used to directly infect a bacterial host cell, such as E. coli ER2738. The infected host cells are grown in an appropriate growth medium, such as LB (Luria-Bertani) medium, and this culture is spread onto agar, containing a suitable growth medium, such as LB medium with IPTG (isopropyl β-D-thiogalactopyranoside) and S-GAL. After growth, the plaques are picked for DNA isolation and sequencing to identify the peptide sequences with a high binding affinity for the hair, skin or nail substrate.

In another embodiment, PCR may be used to identify the elution-resistant phage-peptides from the modified phage display screening method, described above, by directly carrying out PCR on the phage-peptide-substrate complexes using the appropriate primers, as described by Janssen et al. in U.S. Patent Application Publication No. 2003/0152976, which is incorporated herein by reference.

Hair-binding, skin-binding, and nail-binding peptides have been identified using the above methods, as described by Huang et al. in copending and commonly owned U.S. Pat. No. 7,220,405, and U.S. Patent Application Publication No. 2005/0226839, both of which are incorporated herein by reference. Additional hair and skin-binding peptide have been reported in the art (WO 04/048399). Examples of hair-binding peptides are provided herein as SEQ ID NOs: 1, 3-59, 66, 69-73, 75-97, 104-108, 112-116, and 125. Hair-binding peptides reported by Huang et al. in U.S. Patent Application Publication No. 2005/0226839 include those that have a high affinity for hair normal (e.g. brown) hair, given as SEQ ID NOs: 3-18, 28-38, 40-56, and 64; shampoo resistant peptides having affinity for normal brown hair, given as SEQ ID NO:66, 69 and 70; bleached hair, given as SEQ ID NOs: 7, 8, 19-27, 38-40, 43, 44, 47, 57, 58, and 59, fingernail, given as SEQ ID NOs: 53 and 60; and skin, given as SEQ ID NO:61 Additionally, the fingernail-binding peptides were found to bind to bleached hair and may be used in the peptide-based hair reagents of the invention. The bleached hair-binding peptides will bind to fingernails and may be used in the peptide-based nail reagents of the invention.

Alternatively, hair and skin-binding peptide sequences may be generated empirically by designing peptides that comprise positively charged amino acids, which can bind to hair and skin via electrostatic interaction, as described by Rothe et al. (WO 2004/000257). The empirically generated hair and skin-binding peptides have between about 4 amino acids to about 50 amino acids, preferably from about 4 to about 25 amino acids, and comprise at least about 40 mole % positively charged amino acids, such as lysine, arginine, and histidine. Peptide sequences containing tripeptide motifs such as HRK, RHK, HKR, RKH, KRH, KHR, HKX, KRX, RKX, HRX, KHX and RHX are most preferred where X can be any natural amino acid but is most preferably selected from neutral side chain amino acids such as glycine, alanine, proline, leucine, isoleucine, valine and phenylalanine. In addition, it should be understood that the peptide sequences must meet other functional requirements in the end use including solubility, viscosity and compatibility with other components in a formulated product and will therefore vary according to the needs of the application. In some cases the peptide may contain up to 60 mole % of amino acids not comprising histidine, lysine or arginine. Suitable empirically generated hair-binding and skin peptides include, but are not limited to, SEQ ID NOs:104-108.

Preferred hair, skin and nail binding peptides for use in the present invention are SEQ ID NO: 43, 61, 39, 38, and 4, 40, 44, 47, and 53-60.

Production of Binding Peptides

The binding peptides of the present invention may be prepared using standard peptide synthesis methods, which are well known in the art (see for example Stewart et al., Solid Phase Peptide Synthesis, Pierce Chemical Co., Rockford, Ill., 1984; Bodanszky, Principles of Peptide Synthesis, Springer-Verlag, New York, 1984; and Pennington et al., Peptide Synthesis Protocols, Humana Press, Totowa, N.J., 1994). Additionally, many companies offer custom peptide synthesis services.

Alternatively, the peptides of the present invention may be prepared using recombinant DNA and molecular cloning techniques. Genes encoding the hair-binding, skin-binding or nail-binding peptides may be produced in heterologous host cells, particularly in the cells of microbial hosts.

Preferred heterologous host cells for expression of the binding peptides of the present invention are microbial hosts that can be found broadly within the fungal or bacterial families and which grow over a wide range of temperature, pH values, and solvent tolerances. Because transcription, translation, and the protein biosynthetic apparatus are the same irrespective of the cellular feedstock, functional genes are expressed irrespective of carbon feedstock used to generate cellular biomass. Examples of host strains include, but are not limited to, fungal or yeast species such as Aspergillus, Trichoderma, Saccharomyces, Pichia, Candida, Hansenula, or bacterial species such as Salmonella, Bacillus, Acinetobacter, Rhodococcus, Streptomyces, Escherichia, Pseudomonas, Methylomonas, Methylobacter, Alcaligenes, Synechocystis, Anabaena, Thiobacillus, Methanobacterium and Klebsiella.

A variety of expression systems can be used to produce the peptides of the present invention. Such vectors include, but are not limited to, chromosomal, episomal and virus-derived vectors, e.g., vectors derived from bacterial plasmids, from bacteriophage, from transposons, from insertion elements, from yeast episomes, from viruses such as baculoviruses, retroviruses and vectors derived from combinations thereof such as those derived from plasmid and bacteriophage genetic elements, such as cosmids and phagemids. The expression system constructs may contain regulatory regions that regulate as well as engender expression. In general, any system or vector suitable to maintain, propagate or express polynucleotide or polypeptide in a host cell may be used for expression in this regard. Microbial expression systems and expression vectors contain regulatory sequences that direct high level expression of foreign proteins relative to the growth of the host cell. Regulatory sequences are well known to those skilled in the art and examples include, but are not limited to, those which cause the expression of a gene to be turned on or off in response to a chemical or physical stimulus, including the presence of regulatory elements in the vector, for example, enhancer sequences. Any of these could be used to construct chimeric genes for production of the any of the binding peptides of the present invention. These chimeric genes could then be introduced into appropriate microorganisms via transformation to provide high level expression of the peptides.

Vectors or cassettes useful for the transformation of suitable host cells are well known in the art. Typically the vector or cassette contains sequences directing transcription and translation of the relevant gene, one or more selectable markers, and sequences allowing autonomous replication or chromosomal integration. Suitable vectors comprise a region 5′ of the gene, which harbors transcriptional initiation controls and a region 3′ of the DNA fragment which controls transcriptional termination. It is most preferred when both control regions are derived from genes homologous to the transformed host cell, although it is to be understood that such control regions need not be derived from the genes native to the specific species chosen as a production host. Selectable marker genes provide a phenotypic trait for selection of the transformed host cells such as tetracycline or ampicillin resistance in E. coli.

Initiation control regions or promoters which are useful to drive expression of the chimeric gene in the desired host cell are numerous and familiar to those skilled in the art. Virtually any promoter capable of driving the gene is suitable for producing the binding peptides of the present invention including, but not limited to: CYC1, HIS3, GAL1, GAL10, ADH1, PGK, PHO5, GAPDH, ADC1, TRP1, URA3, LEU2, ENO, TPI (useful for expression in Saccharomyces); AOX1 (useful for expression in Pichia); and lac, ara, tet, trp, IP_{L}, IP_{R}, T7, tac, and trc (useful for expression in Escherichia coli) as well as the amy, apr, npr promoters and various phage promoters useful for expression in Bacillus.

Termination control regions may also be derived from various genes native to the preferred hosts. Optionally, a termination site may be unnecessary, however, it is most preferred if included.

The vector containing the appropriate DNA sequence as described supra, as well as an appropriate promoter or control sequence, may be employed to transform an appropriate host to permit the host to express the peptide of the present invention. Cell-free translation systems can also be employed to produce such peptides using RNAs derived from the DNA constructs of the present invention. Optionally it may be desired to produce the instant gene product as a secretion product of the transformed host. Secretion of desired proteins into the growth media has the advantages of simplified and less costly purification procedures. It is well known in the art that secretion signal sequences are often useful in facilitating the active transport of expressible proteins across cell membranes. The creation of a transformed host capable of secretion may be accomplished by the incorporation of a DNA sequence that codes for a secretion signal which is functional in the production host. Methods for choosing appropriate signal sequences are well known in the art (see for example EP 546049 and WO 9324631). The secretion signal DNA or facilitator may be located between the expression-controlling DNA and the instant gene or gene fragment, and in the same reading frame with the latter.

Conditioning Peptides

Any peptide that is believed to produce a conditioning effect on skin hair or nails can be linked to an appropriate body surface binder either directly or indirectly. Conditioners improve the quality of a body surface. Hair conditioners improve the quality of hair by strengthening hair, improving the texture and appearance of hair, protecting hair from damage promoting growth, and providing other benefits. Skin conditioners improve the quality of skin by improving the elasticity of skin, providing a more supple feel to skin, reducing the appearance and effect of age, protecting skin from sunlight and other damaging factors, and providing other benefits. Nail conditioners improve the quality of nail by preventing cracking, strengthening the nail surface, improving the hardness of the nail, promoting nail growth, and providing other benefits. Preferred conditioning peptides include those found naturally, those derived from natural peptides, or those designed or discovered to have conditioning properties. Conditioning peptides that are found in nature that may be used with the present invention include elastin, collagen, abductin, byssus, flagelliform silk, dragline silk, gluten high molecular weight subunit, titin, fibronectin, laminin, gliadin, glue polypolypeptide, ice nucleating protein, keratin, mucin, RNA polymerase II, resilin or a mixture thereof. Examples of repetitively sequenced proteins from which conditioning peptides may be constructed are described in commonly owned U.S. Pat. No. 6,268,169; and U.S. Pat. No. 6,608,242; and Collier et al., US 2004/0234609, all incorporated herein by reference.

Conditioning peptides of the invention are those that are derived from repetitively sequenced proteins. Repetitively sequenced proteins of the present invention are comprised of naturally or non-naturally occurring repeating units. Additionally, synthetic repeating units may be utilized. Individual repeating units of from about 1 unit to about 50 units where repeats will typically comprise from 3 to 50 amino acids, and will usually have the same amino acid appearing at least twice in the same unit. Different unit combinations may be joined together to form a block copolymer or alternating block copolymer.

Individual repeating amino acid sequence units of particular interest include units found in silk, elastin, collagen, abductin, byssus, gluten, titin-, extensin, laminin, and fibronectin-like proteins. Silk-like proteins have a repeating unit of SGAGAG (SEQ ID NO: 126). Elastin-like proteins have a base repeating unit of GVGVP (SEQ ID NO: 127). This repeating unit may be found in naturally occurring elastin. Collagen-like proteins have repeating units of G-X-Y (X=any amino acid, often alanine or proline; Y=any amino acid, often proline or hydroxy-proline). Abductin-like proteins have a base repeating unit of GGFGGMGGGX (F=phenylalanine; M=methionine, X=any amino acid) (SEQ ID NO: 128). Byssus-like proteins have a repeating unit of (GPGGG) (SEQ ID NO: 129). Gluten-like proteins of the high molecular weight subunit have repeating units of PGQGQQ (SEQ ID NO: 130), GYYPTSPQQ (SEQ ID NO: 170), and GQQ (Q=glutamine; Y=tyrosine; T=threonine) SEQ ID NO: 131). Titin-like proteins have a repeating units of PPAKVPEVPKKPVPEEKVPVPVPKKPEA (K=Lysine, E=Glutamic Acid) (SEQ ID NO: 132) and are found in the heart, psoas, and soleus muscle. Extensin-like proteins have repeating units of SPPPPSPKYVYK (SEQ ID NO: 133). Fibronectin-like proteins have repeating units of RGDS (R=arginine; D=aspartic acid) (SEQ ID NO: 134).

Additional repeating units of interest are found in gliadin, glue polypolypeptide (mussel adhesive protein), ice nucleating protein, keratin, mucin, RNA polymerase II, and resilin. Gliadin contains a repeating unit of PQQPY (SEQ ID NO: 135). The glue polypeptide contains a repeating unit of PTTTK (SEQ ID NO: 136). The ice nucleating protein contains a repeating unit of AGYGSTGT (SEQ ID NO: 137). Keratin contains repeating units of YGGSSGGG (SEQ ID NO: 138) or FGGGS (SEQ ID NO. 139). Mucin contains a repeating unit of TTTPDV (SEQ ID NO: 140). RNA polymerase II contains a repeating unit of YSPTSPS (SEQ ID NO: 141). Additionally, resilin, a rubber-like protein contains repeating units.

It will be understood by those having skill in the art that the repeat sequence protein polymers of the present invention may be engineered to include appropriate repeating units in order to provide desired characteristics. For example, the repeat sequence protein polymers may be produced to have moisturizing or conditioning properties. The molecular weight and amino acid composition of the protein may be chosen in order to increase or decrease water solubility as desired.

Repetitively sequenced protein polymers utilizing the natural or synthetic repeating units may have their properties altered by appropriate choice of different units, the number of units in each multimer, the spacing between units, and the number of repeats of the multimer combination assembly. Preferred polymers are combinations of silk units and elastin units to provide silk-elastin polymers having properties distinctive from polymers having only the same monomeric unit.

It will be understood by those having skill in the art that the repeat sequence protein polymers of the present invention may be produced to have a combination of desirable characteristics. For example a polymer having silk repeating units and elastin repeating units may be produced to impart durability due to the silk repeating units and to impart flexibility due to the elastin repeating units. Additionally, the silk-elastin polymer may exhibit other desirable properties such as good clear film and hydrogel formation, which the individual monomeric units may not exhibit. The silk-elastin polymer may be hydrophilic and water soluble. The silk-elastin polymer may have a high isoelectric point which may make the polymer more substantive to skin and hair. The silk-elastin polymer may further exhibit self assembly into fibers and films which may be desirable in some applications.

One preferred embodiment of the invention makes use of silk-like proteins as the repeat sequence protein that serves as the source of the conditioning peptide. Examples of silk-like proteins useful in the present invention are described in commonly owned U.S. Pat. No. 6,608,242 and U.S. Pat. No. 6,268,169, both incorporated herein by reference.

With regard to silk-like proteins, of particular interest are polypeptides which have as a repeating unit SGAGAG (SEQ ID NO: 126) and GAGAGS (SEQ ID NO: 118). This repeating unit is found in a naturally occurring silk fibroin protein, which can be represented as GAGAG(SGAGAG)₈ SGAAGY (SEQ ID NO: 142). Particularly suitable in the present invention are silk-like proteins having the general formula:
[(A)e-(E)f-(S)f-(X)p-(E)f-(S)f]i
wherein:
A or E are different non-crystalline soft segments of about 10 to 25 amino acids having at least 55% Gly;
S is a semi-crystalline segment of about 6 to 12 amino acids having at least 33% Ala, and 50% Gly;
X is a crystalline hard segment of about 6-12 amino acids having at least 33% Ala, and 50% Gly; and
   wherein,
e=2, 4, 8, 16, 32, 64, 128;
f=0, 1, 2, 4, 8, 16, 32, 64, 128;
p=2, 4, 8, 16, 32, 64, 128;
i=1-128; and
   where p≧n or f.

Preferred combinations of the non-crystalline, semi-crystalline or hard segments will include, but are not limited to [(A)₄-(X)₈]₈, [(A)₄-(X)₈-(S)]₈, [(A)₄-(X)₈-(E)]₈, [(A)₈-(X)₈]₈, [(A)₄-(S)-(X)₈]₈, [(A)₄-(S)₂-(X)₈]₈, [(A)₄-(E)-(X)₈-(E)]₈, [(A)₄-(E)-(X)₈]₈, [(A)₄-(S)-(X)₈-(E)]₈, and [(A)₄-(S)₂-(X)₈-(E)]₈. Most preferred combinations are these in which the non-crystalline, semi-crystalline or hard segments are defined as follows: A=SGGAGGAGG (SEQ ID NO: 143), E=GPGQQGPGGY (SEQ ID NO: 144), S=GAGAGY (SEQ ID NO: 145), and X=SGAGAG (SEQ ID NO: 126).

In a preferred embodiment the silk or SLP may be derived form spider silk. There are a variety of spider silks which may be suitable for expression in plants. Many of these are derived from the orb-weaving spiders such as those belonging to the genus Nephila. Silks from these spiders may be divided into major ampullate, minor ampullate, and flagelliform silks, each having different physical properties. For a review of suitable spider silks see Hayashi et al., Int. J. Biol. Macromol. (1999), 24(2,3):271-275, for example. Those of the major ampullate are the most completely characterized and are often referred to as spider dragline silk. Natural spider dragline consists of two different proteins that are co-spun from the spider's major ampullate gland. The amino acid sequence of both dragline proteins has been disclosed by Xu et al., Proc. Natl, Acad. Sci. U.S.A., (1990) 87:7120-7124 and Hinman and Lewis, J. Biol. Chem. (1992) 267:19320-19324, and will be identified hereinafter as Dragline Protein 1 (DP-1) and Dragline Protein 2 (DP-2). Within the context of the present invention Dragline Protein 1 (DP-1) and Dragline Protein 2 (DP-2) were the focus for spider silk variant design.

The design of the spider silk variant proteins is based on consensus amino acid sequences derived from the fiber forming regions of the natural spider silk dragline proteins of Nephila clavipes. The amino acid sequence of a fragment of DP-1 is repetitive and rich in glycine and alanine, but is otherwise unlike any previously known amino acid sequence. The “consensus” sequence of a single repeat, viewed in this way, is:

(SEQ ID NO:146)

AGQGGYGGLGXQGAGRGGLGGQGAGAAAAAAAGG

where X may be S, G, or N.

Individual repeats differ from the consensus according to a pattern which can be generalized as follows: (1) the poly-alanine sequence varies in length from zero to seven residues, (2) when the entire poly-alanine sequence is deleted, so also is the surrounding sequence encompassing AGRGGLGGQGAGAₙGG (SEQ ID NO: 147), (3) aside from the poly-alanine sequence, deletions generally encompass integral multiples of three consecutive residues, (4) deletion of GYG is generally accompanied by deletion of GRG in the same repeat, and (5) a repeat in which the entire poly-alanine sequence is deleted is generally preceded by a repeat containing six alanine residues.

Synthetic analogs of DP-1 were designed to mimic both the repeating consensus sequence of the natural protein and the pattern of variation among individual repeats. Two analogs of DP-1 were designed and designated DP-1A and DP-1B. DP-1A is composed of a tandemly repeated 101-amino acid sequence listed in SEQ ID NO:148. The 101-amino acid “monomer” comprises four repeats which differ according to the pattern (1)-(5) above. This 101-amino acid long peptide monomer is repeated from 1 to 16 times in a series of analog proteins. DP-1B was designed by reordering the four repeats within the monomer of DP-1A. This monomer sequence, shown in SEQ ID NO:149, exhibits all of the regularities of (1)-(5) above. In addition, it exhibits a regularity of the natural sequence which is not shared by DP-1A, namely that a repeat in which both GYG and GRG are deleted is generally preceded by a repeat lacking the entire poly-alanine sequence, with one intervening repeat. The sequence of DP-1B matches the natural sequence more closely over a more extended segment than does DP-1A.

Thus it is an object of the present invention to provide a spider dragline variant protein wherein the full length variant protein is defined by the formula:

(SEQ ID NO:150-157)

[ACGQGGYGGLGXQGAGRGGLGGQGAGA_{g}GG]ₕ

wherein X=S, G or N; g=0-7 and h=1-75, and wherein the value of z determines the number of repeats in the variant protein and wherein the formula encompasses variations selected from the group consisting of:
(a) when g=0 the sequence encompassing AGRGGLGGQGAGAₙGG (SEQ ID NO:147) is deleted;
(b) deletions other than the poly-alanine sequence, limited by the value of n will encompass integral multiples of three consecutive residues;
(c) the deletion of GYG in any repeat is accompanied by deletion of GRG in the same repeat; and
(d) where a first repeat where g=0 is deleted, the first repeat is preceded by a second repeat where g=6; and
   wherein the full-length protein is encoded by a gene or genes and wherein said gene or genes are not endogenous to the Nephila clavipes genome.

The silk variants and SLP's of the present invention will have physical properties commonly associated with natural proteins. So for example, the silks and SLP's will be expected to have tenacities (g/denier) of about 2.8 to about 5.2, tensile strengths (psi) of about 45,000 to about 83,000 and elongations (%) of about 13 to about 31.

In one embodiment, the conditioning peptide comprises at least one peptide having an amino acid sequence selected from the group consisting of SEQ ID NOs: 117-122, 126-158, 160, 162,164-165, 170, and 173-197.

Peptide-Based Conditioning Reagents

The peptide-based body surface conditioning reagents of the present invention are formed by coupling at least one body surface-binding peptide to at least one conditioning peptide, either directly or through a molecular spacer. Preferable body surface-binding peptides are those that bind selectively to hair, skin and nails. The body surface-binding peptide part of the reagent binds strongly to the body surface, thereby attaching the conditioning peptide to the body surface. The peptide-based body surface conditioning reagents of the invention are from about 14 to about 200 amino acids in length, preferably about 30 to about 130 amino acids in length, and are typically less than about 200,000 Daltons in molecular weight.

Suitable body surface-binding peptides are described above and include, but are not limited to hair-binding, skin-binding, and nail-binding, peptides selected by the screening methods described above, and empirically generated hair and skin-binding peptides, as described above. Additionally, any known body surface-binding peptide may be used, including hair-binding peptides such as SEQ ID NO:1, and skin-binding peptides such as SEQ ID NO:2, described by Janssen et al. in U.S. Patent Application Publication No. 2003/0152976, and hair-binding peptides such as SEQ ID NOs:75-97, and skin-binding peptides such as SEQ ID NOs:98-103, described by Janssen et al. in WO 04048399, both of which are incorporated herein by reference. Additionally, hair conditioner resistant hair-binding peptides such as SEQ ID NO:112, described by Wang et al. (U.S. Patent Application Publication No. 2007/0196305), and hair conditioner and shampoo resistant hair-binding peptides such as SEQ ID NOs:112-115, as described by O'Brien et al. (U.S. Patent Application Publication No. 2006/0073111), may be used. Suitable conditioning peptides are those described above.

The peptide-based body surface conditioning reagents of the present invention are prepared by coupling at least one body surface-binding peptide to at least one conditioning peptide, either directly or via an optional spacer. The coupling interaction may be a covalent bond or a non-covalent interaction, such as hydrogen bonding, electrostatic interaction, hydrophobic interaction, or Van der Waals interaction. In the case of a non-covalent interaction, the peptide-based body surface conditioning reagents may be prepared by mixing at least one body surface-binding peptide, at least one conditioning peptide and the optional spacer (if used) and allowing sufficient time for the interaction to occur. The unbound materials may be separated from the resulting peptide-based body surface conditioning reagent using methods known in the art, for example, gel permeation chromatography.

The peptide-based body surface conditioning reagents of the invention may also be prepared by covalently attaching at least one body surface-binding peptide to at least one conditioning peptide, either directly or through a spacer. Any known peptide or protein conjugation chemistry may be used to form the peptide-based body surface conditioning reagents of the invention. Conjugation chemistries are well-known in the art (see for example, Hermanson, Bioconjugate Techniques, Academic Press, San Diego, Calif. (1996)). Suitable coupling agents include, but are not limited to, carbodiimide coupling agents, diacid chlorides, diisocyanates and other difunctional coupling reagents that are reactive toward terminal amine and/or carboxylic acid groups on the peptides. The preferred coupling agents are carbodiimide coupling agents, such as 1-ethyl-3-(3-dimethylaminopropyl)-carbodiimide (EDC) and N,N′-dicyclohexyl-carbodiimide (DCC), which may be used to activate carboxylic acid groups. Additionally, it may be necessary to protect reactive amine or carboxylic acid groups on the peptides to produce the desired structure for the peptide-based body surface conditioning reagent. The use of protecting groups for amino acids, such as t-butyloxycarbonyl (t-Boc), are well known in the art (see for example Stewart et al., supra; Bodanszky, supra; and Pennington et al., supra).

Additionally, peptide-based body surface conditioning reagents consisting of at least one body surface-binding peptide and at least one conditioning peptide may be prepared using the recombinant DNA and molecular cloning techniques described supra.

It may also be desirable to couple the body surface-binding peptide to the conditioning peptide via a spacer to form a triblock body surface conditioning reagent. The spacer serves to separate the binding peptide sequences to ensure that the binding affinity of the individual peptides is not adversely affected by the coupling. The spacer may also provide other desirable properties such as hydrophilicity, hydrophobicity, or a means for cleaving the peptide sequences to facilitate removal of the conditioning peptide.

The spacer may be any of a variety of molecules, such as alkyl chains, phenyl compounds, ethylene glycol, amides, esters and the like. Preferred spacers are hydrophilic and have a chain length from 1 to about 100 atoms, more preferably, from 2 to about 30 atoms. Examples of preferred spacers include, but are not limited to ethanol amine, ethylene glycol, polyethylene with a chain length of 6 carbon atoms, polyethylene glycol with 3 to 6 repeating units, phenoxyethanol, propanolamide, butylene glycol, butyleneglycolamide, propyl phenyl chains, and ethyl, propyl, hexyl, steryl, cetyl, and palmitoyl alkyl chains. The spacer may be covalently attached to the body surface-binding and conditioning peptide sequences using any of the coupling chemistries described above. In order to facilitate incorporation of the spacer, a bifunctional coupling agent that contains a spacer and reactive groups at both ends for coupling to the peptides may be used. Suitable bifunctional coupling agents are well known in the art and include, but are not limited to diamines, such as 1,6-diaminohexane; dialdehydes, such as glutaraldehyde; bis N-hydroxysuccinimide esters, such as ethylene glycol-bis(succinic acid N-hydroxysuccinimide ester), disuccinimidyl glutarate, disuccinimidyl suberate, and ethylene glycol-bis(succinimidylsuccinate); diisocyanates, such as hexamethylenediisocyanate; bis oxiranes, such as 1,4 butanediyl diglycidyl ether; dicarboxylic acids, such as succinyldisalicylate; and the like. Heterobifunctional coupling agents, which contain a different reactive group at each end, may also be used. Examples of heterobifunctional coupling agents include, but are not limited to compounds having the following structure: 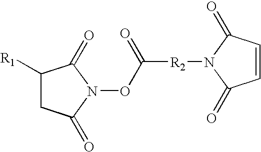
where: R₁ is H or a substituent group such as —SO₃Na, —NO₂, or —Br; and R₂ is a spacer such as —CH₂CH₂ (ethyl), —(CH₂)₃ (propyl), or —(CH₂)₃C₆H₅ (propyl phenyl). An example of such a heterobifunctional coupling agent is 3-maleimidopropionic acid N-hydroxysuccinimide ester. The N-hydroxysuccinimide ester group of these reagents reacts with amine groups on one peptide, while the maleimide group reacts with thiol groups present on the other peptide. A thiol group may be incorporated into the peptide by adding at least one cysteine group to at least one end of the binding peptide sequence (i.e., the C-terminal end or N-terminal end). Several spacer amino acid residues, such as glycine, may be incorporated between the binding peptide sequence and the terminal cysteine to separate the reacting thiol group from the binding sequence. Moreover, at least one lysine residue may be added to at least one end of the binding peptide sequence, i.e., the C-terminal end or the N-terminal end, to provide an amine group for coupling.

Additionally, the spacer may be a peptide comprising any amino acid and mixtures thereof. The preferred peptide spacers comprise the amino acids proline, lysine, glycine, alanine, and serine, and mixtures thereof. In addition, the peptide spacer may contain a specific enzyme cleavage site, such as the protease Caspase 3 site, given by SEQ ID NO:65, which allows for the enzymatic removal of pigment from the hair. The peptide spacer may be from 2 to about 50 amino acids, preferably from 1 to about 20 amino acids in length. Examples of suitable spacers include, but are not limited to, the sequences given by SEQ ID NOs:109-111, 123-124, and 159. These peptide spacers may be linked to the binding peptide sequences by any method known in the art. For example, the entire triblock peptide-based body surface conditioning reagent may be prepared using the standard peptide synthesis methods described supra. In addition, the binding peptides and peptide spacer block may be combined using carbodiimide coupling agents (see for example, Hermanson, Bioconjugate Techniques, Academic Press, San Diego, Calif. (1996)), diacid chlorides, diisocyanates and other difunctional coupling reagents that are reactive to terminal amine and/or carboxylic acid groups on the peptides, as described above. Alternatively, the entire triblock peptide-based body surface conditioning reagent may be prepared using the recombinant DNA and molecular cloning techniques described supra. The spacer may also be a combination of a peptide spacer and an organic spacer molecule, which may be prepared using the methods described above. Examples of body surface peptide-based conditioning reagents include, but are not limited to the sequences given as SEQ ID NOs: 161, 163, and 166.

It may also be desirable to have multiple copies of the body surface-binding peptide and the conditioning peptide coupled together to enhance the interaction between the peptide-based body surface conditioning reagent and the body surface, as described by Huang et al. (U.S. Pat. No. 7,220,405 and U.S. Patent Application Publication No. 2005/0226839). Either multiple copies of the same body surface-binding peptide and conditioning peptide or a combination of different body surface-binding peptides and conditioning peptides may be used. The multi-copy peptide-based body surface conditioning reagents may comprise various spacers as described above.

In one embodiment of the invention, the peptide-based body surface conditioning reagent is a diblock composition comprising a body surface-binding peptide (BSBP) and a conditioning peptide (CP), having the general structure [(BSBP)ₘ-(CP)ₙ]ₓ, where n and m independently range from 1 to about 10, preferably from 1 to about 5, and x may be 1 to about 10. In a preferred embodiment the diblock conditioning reagent has a molecular weight of less than about 200,000 Daltons.

In another embodiment, the peptide-based body surface conditioning reagent comprises a molecular spacer (S) separating the body surface-binding peptide from the conditioning peptide, as described above. Multiple copies of the body surface-binding peptide and the conditioning peptide may also be used and the multiple copies of the body surface-binding peptide and the conditioning peptide may be separated from themselves and from each other by molecular spacers. In this embodiment, the peptide-based body surface conditioning reagent is a triblock composition comprising a body surface-binding peptide, a spacer, and conditioning peptide, having the general structure [[(BSBP)ₘ-S_{q}]ₓ-[(CP)ₙ-Sᵣ]_{z}]_{y}, where n, m, x, and z independently range from 1 to about 10, y is from 1 to about 5, and where q and r are each independently 0 or 1. Preferably, m and n independently range from 1 to about 5, and x and z range from 1 to about 3. In a preferred embodiment the triblock conditioning reagent has a molecular weight of less than about 200,000 Daltons.

In another embodiment, the body surface-binding peptide is a hair-binding peptide and the peptide-based body surface conditioning reagent is a diblock composition comprising the hair-binding peptide (HBP) and a conditioning peptide (CP), having the general structure [(HBP)ₘ-(CP)ₙ]ₓ where n and m independently range from 1 to about 10, preferably from 1 to about 5, and x may be 1 to about 10.

In another embodiment, the body surface-binding peptide is a hair-binding peptide and the peptide-based body surface conditioning reagent is a triblock composition comprising the hair-binding peptide (HBP), a spacer (S), and a conditioning peptide (CP), having the general structure [[(HBP)ₘ-S_{q}]ₓ-[(CP)ₙ-Sᵣ]_{z}]_{y}, where n, m, x, and z independently range from 1 to about 10, y is from 1 to about 5, and where q and r are each independently 0 or 1. Preferably, m and n independently range from 1 to about 5, and x and z range from 1 to about 3.

In another embodiment, the body surface-binding peptide is a skin-binding peptide and the peptide-based body surface conditioning reagent is a diblock composition comprising the skin-binding peptide (SBP) and a conditioning peptide (CP), having the general structure [(SBP)ₘ-(CP)ₙ]ₓ, where n and m independently range from 1 to about 10, preferably from 1 to about 5, and x may be 1 to about 10.

In another embodiment, the body surface-binding peptide is a skin-binding peptide and the peptide-based body surface conditioning reagent is a triblock composition comprising the skin-binding peptide (SBP), a spacer (S), and a conditioning peptide (CP), having the general structure [[(SBP)ₘ-S_{q}]ₓ-[(CP)ₙ-Sᵣ]_{z}]_{y}, where n, m, x, and z independently range from 1 to about 10, y is from 1 to about 5, and where q and r are each independently 0 or 1. Preferably, m and n independently range from 1 to about 5, and x and z range from 1 to about 3.

In another embodiment, the body surface-binding peptide is a nail-binding peptide and the peptide-based body surface conditioning reagent is a diblock composition comprising the nail-binding peptide (NBP) and a conditioning peptide (CP), having the general structure [(NBP)ₘ-(CP)ₙ]ₓ where n and m independently range from 1 to about 10, preferably from 1 to about 5, and x may be 1 to about 10.

In another embodiment, the body surface-binding peptide is a nail-binding peptide and the peptide-based body surface conditioning reagent is a triblock composition comprising the nail-binding peptide (NBP), a spacer (S), and a conditioning peptide (CP), having the general structure [[(NBP)ₘ-S_{q}]ₓ-[(CP)ₙ-Sᵣ]_{z}]_{y}, where n, m, x, and z independently range from 1 to about 10, y is from 1 to about 5, and where q and r are each independently 0 or 1. Preferably, m and n independently range from 1 to about 5, and x and z range from 1 to about 3.

It should be understood that as used herein, BSBP, HBP, SBP, NBP, and CP are generic designations and are not meant to refer to a single body surface-binding peptide, hair-binding peptide, skin-binding peptide, nail-binding peptide, or conditioning peptide sequence, respectively. Where m or n as used above, is greater than 1, it is well within the scope of the invention to provide for the situation where a series of body surface-binding peptides of different sequences and conditioning peptides of different sequences may form a part of the composition. Additionally, S is a generic term and is not meant to refer to a single spacer. Where x and y, as used above for the triblock compositions, are greater than 1, it is well within the scope of the invention to provide for the situation where a series of different spacers may form a part of the composition. It should also be understood that these structures do not necessarily represent a covalent bond between the peptides and the optional molecular spacer. As described above, the coupling interaction between the peptides and the optional spacer may be either covalent or non-covalent.

The above description recites the parameters around which peptide-based conditioning reagents of the invention are designed and constructed. The following Table B lists preferred examples of combinations of body surface-binding peptides, spacers and conditioning peptides that may be combined in any manner to produce the conditioning reagents of the invention.

TABLE B

| | | | SEQ | | SEQ | Conditioning | SEQ |
|---|---|---|---|---|---|---|---|
| BSBP | Body | | ID | | ID | Peptide | ID |
| ID | Surface | Sequence | NO: | Spacer | NO | Repeat | NO |
| F01 | Nail | ALPRIANTWSPS | 60 | GGP | 123 | SGGAGGAGG | 143 |
| D05 | Nail | YPSFSPTYRPAF | 53 | GPGVG | 124 | GPGQQGPGGY | 144 |
| D39 | Hair | LGIPQNL | 39 | | | GAGAGY | 119 |
| B1 | Hair | TAATTSP | 38 | | | SGAGAG | 126 |
| A5 | Hair | EQISGSLVAAPW | 43 | | | GAGAGS | 118 |
| C4 | Hair | NEVPARNAPWLV | 57 | | | GVGVP | 127 |
| D30 | Hair | NSPGYQADSVAIG | 58 | | | GGFGGMGGGX | 128 |
| C44 | Hair | AKPISQHLQRGS | 40 | | | GPGGG | 129 |
| E66 | Hair | LDTSFPPVPFHA | 44 | | | PGQGQQ | 130 |
| C45 | Hair | SLNWVTIPGPKI | 47 | | | GYYPTSPQQ | 170 |
| E18 | Hair | TQDSAQKSPSPL | 59 | | | GQQ | 131 |
| I-B5 | Hair | TPPELLHGDPRS | 66 | | | PPAKVPEVPKKPVPEEKVPVPVPKKPEA | 132 |
| SK-1 | Skin | TPFHSPENAPGS | 61 | | | SPPPPSPKYVYK | 133 |
| | | | | | | PQQPY | 135 |
| | | | | | | PTTTK | 136 |
| | | | | | | AGYGSTGT | 137 |
| | | | | | | YGGSSGGG | 138 |
| | | | | | | FGGGS | 139 |
| | | | | | | TTTPDV | 140 |
| | | | | | | YSPTSPS | 141 |
| | | | | | | KGAGAGAPGAGAGAK | 158 |

Personal Care Conditioning Compositions

The peptide-based body surface conditioning reagents of the invention may be used in personal care compositions to condition body surfaces, such as hair, skin, and nails. The body surface-binding peptide block of the peptide-based body surface conditioning reagent has an affinity for the body surface, while the conditioning peptide block has a film forming function conveying a silky or smooth texture to the body surface. Personal care compositions include, but are not limited to, hair care compositions, skin care compositions, cosmetic compositions, and nail polish compositions.

Hair Care Compositions

In one embodiment, the peptide-based body surface conditioning reagent is a component of a hair care composition and the peptide-based body surface conditioning reagent comprises at least one hair-binding peptide. Hair care compositions are herein defined as compositions for the treatment of hair including, but not limited to, shampoos, conditioners, rinses, lotions, aerosols, gels, mousses, and colorants. An effective amount of the peptide-based body surface conditioning reagent for use in hair care compositions is a concentration of about 0.01% to about 10%, preferably about 0.01% to about 5% by weight relative to the total weight of the composition. This proportion may vary as a function of the type of hair care composition.

Additionally, a mixture of different peptide-based conditioning reagents may be used in the composition. The peptide-based conditioning reagents in the mixture need to be chosen so that there is no interaction between the peptides that mitigates the beneficial effect. Suitable mixtures of peptide-based body surface conditioning reagents may be determined by one skilled in the art using routine experimentation. If a mixture of peptide-based body surface conditioning reagents is used in the composition, the total concentration of the reagents is about 0.01% to about 10% by weight relative to the total weight of the composition.

The composition may further comprise a cosmetically acceptable medium for hair care compositions, examples of which are described by Philippe et al. in U.S. Pat. No. 6,280,747, and by Omura et al. in U.S. Pat. No. 6,139,851 and Cannell et al. in U.S. Pat. No. 6,013,250, all of which are incorporated herein by reference. For example, these hair care compositions can be aqueous, alcoholic or aqueous-alcoholic solutions, the alcohol preferably being ethanol or isopropanol, in a proportion of from about 1 to about 75% by weight relative to the total weight for the aqueous-alcoholic solutions. Additionally, the hair care compositions may contain one or more conventional cosmetic or dermatological additives or adjuvants including, but not limited to, antioxidants, preserving agents, fillers, surfactants, UVA and/or UVB sunscreens, fragrances, thickeners, wetting agents and anionic, nonionic or amphoteric polymers, and dyes.

Skin Care Conditioning Compositions

In another embodiment, the peptide-based body surface conditioning reagent is a component of a skin care composition and the peptide-based body surface conditioning reagent comprises at least one skin-binding peptide. Skin care compositions are herein defined as compositions for the treatment of skin including, but not limited to, skin care, skin cleansing, make-up, and anti-wrinkle products. An effective amount of the peptide-based body surface conditioning reagent for use in a skin care composition is a concentration of about 0.01% to about 10%, preferably about 0.01% to about 5% by weight relative to the total weight of the composition. This proportion may vary as a function of the type of skin care composition. Additionally, a mixture of different peptide-based body surface conditioning reagents may be used in the composition. The peptide-based body surface conditioning reagents in the mixture need to be chosen so that there is no interaction between the peptides that mitigates the beneficial effect. Suitable mixtures of peptide-based body surface conditioning reagents may be determined by one skilled in the art using routine experimentation. If a mixture of peptide-based body surface conditioning reagents is used in the composition, the total concentration of the reagents is about 0.01% to about 10% by weight relative to the total weight of the composition.

The composition may further comprise a cosmetically acceptable medium for skin care compositions, examples of which are described by Philippe et al. supra. For example, the cosmetically acceptable medium may be an anhydrous composition containing a fatty substance in a proportion generally of from about 10 to about 90% by weight relative to the total weight of the composition, where the fatty phase contains at least one liquid, solid or semi-solid fatty substance. The fatty substance includes, but is not limited to oils, waxes, gums, and so-called pasty fatty substances. Alternatively, the compositions may be in the form of a stable dispersion such as a water-in-oil or oil-in-water emulsion. Additionally, the compositions may contain one or more conventional cosmetic or dermatological additives or adjuvants including, but not limited to, antioxidants, preserving agents, fillers, surfactants, UVA and/or UVB sunscreens, fragrances, thickeners, wetting agents and anionic, nonionic or amphoteric polymers, and dyes.

Nail Polish Conditioning Compositions

In another embodiment, the peptide-based body surface conditioning reagent is a component of a nail polish composition and the peptide-based body surface conditioning reagent comprises at least one nail-binding peptide. The nail polish compositions are used for coloring fingernails and toenails and comprise one or more coloring agents.

An effective amount of a peptide-based body surface conditioning reagent for use in a nail polish composition is herein defined as a proportion of from about 0.01% to about 20% by weight relative to the total weight of the composition. Additionally, a mixture of different peptide-based body surface conditioning reagents may be used in the composition. The peptide-based body surface conditioning reagents in the mixture need to be chosen so that there is no interaction between the peptides that mitigates the beneficial effect. Suitable mixtures of peptide-based body surface conditioning reagents may be determined by one skilled in the art using routine experimentation. If a mixture of peptide-based body surface conditioning reagents is used in the composition, the total concentration of the reagents is about 0.01% to about 20% by weight relative to the total weight of the composition.

Components of a cosmetically acceptable medium for nail polish compositions are described by Philippe et al. supra. The nail polish composition typically contains a solvent and a film forming substance, such as cellulose derivatives, polyvinyl derivatives, acrylic polymers or copolymers, vinyl copolymers and polyester polymers. Additionally, the nail polish may contain a plasticizer, such as tricresyl phosphate, benzyl benzoate, tributyl phosphate, butyl acetyl ricinoleate, triethyl citrate, tributyl acetyl citrate, dibutyl phthalate or camphor.

Methods for Treating Hair, Skin, and Nails

In another embodiment, methods are provided for treating hair, skin and nails with the peptide-based body surface conditioning reagent of the present invention. Specifically, the present invention also comprises a method for forming a protective film of conditioning peptides on skin, hair, or nails by applying one of the compositions described above comprising an effective amount of a peptide-based body surface conditioning reagent to the skin, hair, or nails and allowing the formation of the protective film. The compositions of the present invention may be applied to the skin, hair or nails by various means, including, but not limited to spraying, brushing, and applying by hand. The peptide-based body surface conditioning reagent composition is left in contact with the skin, hair, or nails for a period of time sufficient to form the protective film, preferably for at least about 0.1 to 60 min.

### EXAMPLES

The present invention is further defined in the following Examples. It should be understood that these Examples, while indicating preferred embodiments of the invention, are given by way of illustration only. From the above discussion and these Examples, one skilled in the art can ascertain the essential characteristics of this invention, and without departing from the spirit and scope thereof, can make various changes and modifications of the invention to adapt it to various uses and conditions.

The meaning of abbreviations used is as follows: “min” means minute(s), “sec” means second(s), “h” means hour(s), “μL” means microliter(s), “mL” means milliliter(s), “L” means liter(s), “nm” means nanometer(s), “mm” means millimeter(s), “cm” means centimeter(s), “μm” means micrometer(s), “mM” means millimolar, “M” means molar, “mmol” means millimole(s), “μmol” means micromole(s), “g” means gram(s), “μg” means microgram(s), “mg” means milligram(s), “g” means the gravitation constant, “rpm” means revolutions per minute, “pfu” means plague forming unit, “BSA” means bovine serum albumin, “ELISA” means enzyme linked immunosorbent assay, “IPTG” means isopropyl β-D-thiogalactopyranoside, “A” means absorbance, “A₄₅₀” means the absorbance measured at a wavelength of 450 nm, “OD₆₀₀” means the optical density measured at 600 nanometers, “TBS” means Tris-buffered saline, “TBST-X” means Tris-buffered saline containing TWEEN® 20 where “X” is the weight percent of TWEEN® 20, “Xgal” means 5-bromo-4-chloro-3-indolyl-beta-D-galactopyranoside, “SEM” means standard error of the mean, “MW” means molecular weight, “M_{w}” means weight-average molecular weight, “vol %” means volume percent, “wt %” means weight percent, “MALDI mass spectrometry” means matrix assisted, laser desorption ionization mass spectrometry.

General Methods:

Standard recombinant DNA and molecular cloning techniques used herein are well known in the art and are described by Sambrook, J. and Russell, D., Molecular Cloning: A Laboratory Manual, Third Edition, Cold Spring Harbor Laboratory Press, Cold Spring Harbor, N.Y. (2001); and by Silhavy, T. J., Bennan, M. L. and Enquist, L. W., Experiments with Gene Fusions, Cold Spring Harbor Laboratory Cold Press Spring Harbor, N.Y. (1984); and by Ausubel, F. M. et. al., Short Protocols in Molecular Biology, 5^{th} Ed. Current Protocols and John Wiley and Sons, Inc., N.Y., 2002.

Materials and methods suitable for the maintenance and growth of bacterial cultures are also well known in the art. Techniques suitable for use in the following Examples may be found in Manual of Methods for General Bacteriology, Phillipp Gerhardt, R. G. E. Murray, Ralph N. Costilow, Eugene W. Nester, Willis A. Wood, Noel R. Krieg and G. Briggs Phillips, eds., American Society for Microbiology, Washington, D.C., 1994, or by Thomas D. Brock in Biotechnology: A Textbook of Industrial Microbiology, Second Edition, Sinauer Associates, Inc., Sunderland, Mass., 1989. All reagents, restriction enzymes and materials used for the growth and maintenance of bacterial cells were obtained from Aldrich Chemicals (Milwaukee, Wis.), BD Diagnostic Systems (Sparks, Md.), Life Technologies (Rockville, Md.), or Sigma Chemical Company (St. Louis, Mo.), unless otherwise specified.

### Example 1

### Selection of Hair-Binding Phage Peptides Using Standard Biopanning

The purpose of this Example was to identify hair-binding phage peptides that bind to normal hair and to bleached hair using standard phage display biopanning.

Phage Display Peptide Libraries:

The phage libraries used in the present invention, Ph.D.-12™ Phage Display Peptide Library Kit and Ph.D.-7™ Phage Display Library Kit, were purchased from New England BioLabs (Beverly, Mass.). These kits are based on a combinatorial library of random peptide 7 or 12-mers fused to a minor coat protein (pill) of M13 phage. The displayed peptide is expressed at the N-terminus of pill, such that after the signal peptide is cleaved, the first residue of the coat protein is the first residue of the displayed peptide. The Ph.D.-7 and Ph.D.-12 libraries consist of approximately 2.8×10⁹ and 2.7×10⁹ sequences, respectively. A volume of 10 μL contains about 55 copies of each peptide sequence. Each initial round of experiments was carried out using the original library provided by the manufacturer in order to avoid introducing any bias into the results.

Preparation of Hair Samples:

The samples used as normal hair were 6-inch medium brown human hairs obtained from International Hair Importers and Products (Bellerose, N.Y.). The hairs were placed in 90% isopropanol for 30 min at room temperature and then washed 5 times for 10 min each with deionized water. The hairs were air-dried overnight at room temperature.

To prepare the bleached hair samples, the medium brown human hairs were placed in 6% H₂O₂, which was adjusted to pH 10.2 with ammonium hydroxide, for 10 min at room temperature and then washed 5 times for 10 min each with deionized water. The hairs were air-dried overnight at room temperature.

The normal and bleached hair samples were cut into 0.5 to 1 cm lengths and about 5 to 10 mg of the hairs was placed into wells of a custom 24-well biopanning apparatus that had a pig skin bottom. An equal number of the pig skin bottom wells were left empty. The pig skin bottom apparatus was used as a subtractive procedure to remove phage-peptides that have an affinity for skin. This apparatus was created by modifying a dot blot apparatus (obtained from Schleicher & Schuell, Keene, N.H.) to fit the biopanning process. Specifically, the top 96-well block of the dot blot apparatus was replaced by a 24-well block. A 4×6 inch treated pig skin was placed under the 24-well block and panning wells with a pig skin bottom were formed by tightening the apparatus. The pig skin was purchased from a local supermarket and stored at −80° C. Before use, the skin was placed in deionized water to thaw, and then blotted dry using a paper towel. The surface of the skin was wiped with 90% isopropanol, and then rinsed with deionized water. The 24-well apparatus was filled with blocking buffer consisting of 1 mg/mL BSA in TBST containing 0.5% TWEEN® 20 (TBST-0.5%) and incubated for 1 h at 4° C. The wells and hairs were washed 5 times with TBST-0.5%. One milliliter of TBST-0.5% containing 1 mg/mL BSA (bovine serum albumin) was added to each well. Then, 10 μL of the original phage library (2×10¹¹ pfu), either the 12-mer or 7-mer library, was added to the pig skin bottom wells that did not contain a hair sample and the phage library was incubated for 15 min at room temperature. The unbound phage were then transferred to pig skin bottom wells containing the hair samples and were incubated for 15 min at room temperature. The hair samples and the wells were washed 10 times with TBST-0.5%. The hairs were then transferred to clean, plastic bottom wells of a 24-well plate and 1 mL of a non-specific elution buffer consisting of 1 mg/mL BSA in 0.2 M glycine-HCl, pH 2.2, was added to each well and incubated for 10 min to elute the bound phage. Then, 160 μL of neutralization buffer consisting of 1 M Tris-HCl, pH 9.2, was added to each well. The eluted phage from each well were transferred to a new tube for titering and sequencing.

To titer the bound phage, the eluted phage was diluted with SM buffer (100 mM NaCl, 12.3 mM MgSO₄-7H₂O, 50 mM Tris-HCl, pH 7.5, and 0.01 wt/vol % gelatin) to prepare 10-fold serial dilutions of 10¹ to 10⁴. A 10 μL aliquot of each dilution was incubated with 200 μL of mid-log phase E. coli ER2738 (New England BioLabs), grown in LB medium for 20 min and then mixed with 3 mL of agarose top (LB medium with 5 mM MgCl₂, and 0.7% agarose) at 45° C. This mixture was spread onto a S-GAL®/LB agar plate (Sigma Chemical Co.) and incubated overnight at 37° C. The S-GAL®/LB agar blend contained 5 g of tryptone, 2.5 g of yeast extract, 5 g of sodium chloride, 6 g of agar, 150 mg of 3,4-cyclohexenoesculetin-β-D-galactopyranoside (S-GAL®), 250 mg of ferric ammonium citrate and 15 mg of isopropyl β-D-thiogalactoside (IPTG) in 500 mL of distilled water. The plates were prepared by autoclaving the S-GAL®/LB for 15 to 20 min at 121-124° C. The single black plaques were randomly picked for DNA isolation and sequence analysis.

The remaining eluted phage were amplified by incubating with diluted E. coli ER2738, from an overnight culture diluted 1:100 in LB medium, at 37° C. for 4.5 h. After this time, the cell culture was centrifuged for 30 s and the upper 80% of the supernatant was transferred to a fresh tube, 1/6 volume of PEG/NaCl (20% polyethylene glycol-800, 2.5 M sodium chloride) was added, and the phage was allowed to precipitate overnight at 4° C. The precipitate was collected by centrifugation at 10,000×g at 4° C. and the resulting pellet was resuspended in 1 mL of TBS. This was the first round of amplified stock. The amplified first round phage stock was then titered according to the same method as described above. For the next round of biopanning, more than 2×10¹¹ pfu of phage stock from the first round was used. The biopanning process was repeated for 3 to 6 rounds depending on the experiments.

The single plaque lysates were prepared following the manufacture's instructions (New England BioLabs) and the single stranded phage genomic DNA was purified using the QIAprep Spin M13 Kit (Qiagen, Valencia, Calif.) and sequenced at the DuPont Sequencing Facility using −−96 gIII sequencing primer (5′-CCCTCATAGTTAGCGTAACG-3′), given as SEQ ID NO: 62. The displayed peptide is located immediately after the signal peptide of gene III.

The amino acid sequences of the eluted normal hair-binding phage peptides from the 12-mer library isolated from the fifth round of biopanning are given in Table 1. The amino acid sequences of the eluted bleached hair-binding phage peptides from the 12-mer library isolated from the fifth round of biopanning are given in Table 2. Repeated amino acid sequences of the eluted normal hair-binding phage peptides from the 7-mer library from 95 randomly selected clones, isolated from the third round of biopanning, are given in Table 3.

TABLE 1

Amino Acid Sequences of Fluted Normal Hair-
Binding Phage Peptides from 12-Mer Library

| | | | |
|---|---|---|---|
| | | SEQ | |
| Clone | Amino Acid | ID | |
| D | Sequence | NO: | Frequency¹ |

| | | | |
|---|---|---|---|
| 1 | RVPNKTVTVDGA | 5 | 5 |
| 2 | DRHKSKYSSTKS | 6 | 2 |
| 3 | KNFPQQKEFPLS | 7 | 2 |
| 4 | QRNSPPAMSRRD | 8 | 2 |
| 5 | TRKPNMPHGQYL | 9 | 2 |
| 6 | KPPHLAKLPFTT | 10 | 1 |
| 7 | NKRPPTSHRIHA | 11 | 1 |
| 8 | NLPRYQPPCKPL | 12 | 1 |
| 9 | RPPWKKPIPPSE | 13 | 1 |
| 10 | RQRPKDHFFSRP | 14 | 1 |
| 11 | SVPNKXVTVDGX | 15 | 1 |
| 12 | TTKWRHRAPVSP | 16 | 1 |
| 13 | WLGKNRIKPRAS | 17 | 1 |
| 14 | SNFKTPLPLTQS | 18 | 1 |
| 15 | SVSVGMKPSPRP | 3 | 1 |

¹The frequency represents the number of identical sequences that occurred out of 23 sequenced clones.

TABLE 2

Amino Acid Sequences of Eluted Bleached Hair-
Binding Phage Peptides from 12-Mer Library

| | | | |
|---|---|---|---|
| | | SEQ | |
| Clone | Amino Acid | ID | |
| ID | Sequence | NO: | Frequency¹ |

| | | | |
|---|---|---|---|
| 1 | KELQTRNVVQRE | 19 | 8 |
| 2 | QRNSPPAMSRRD | 8 | 5 |
| 3 | TPTANQFTQSVP | 20 | 2 |
| 4 | AAGLSQKHERNR | 21 | 2 |
| 5 | ETVHQTPLSDRP | 22 | 1 |
| 6 | KNFPQQKEFPLS | 7 | 1 |
| 7 | LPALHIQRHPRM | 23 | 1 |
| 8 | QPSHSQSHNLRS | 24 | 1 |
| 9 | RGSQKSKPPRPP | 25 | 1 |
| 10 | THTQKTPLLYYH | 26 | 1 |
| 11 | TKGSSQAILKST | 27 | 1 |

¹The frequency represents the number of identical sequences that occurred out of 24 sequenced clones.

TABLE 3

Amino Acid Sequences of Fluted Normal Hair-
Binding Phage Peptides from 7-Mer Library

| | | SEQ |
|---|---|---|
| Clone | | ID |
| ID | Amino Acid Sequence | NO: |
| A | DLHTVYH | 28 |
| B | HIKPPTR | 29 |
| D | HPVWPAI | 30 |
| E | MPLYYLQ | 31 |
| F¹ | HLTVPWRGGGSAVPFYSHSQITLPNH | 32 |
| G¹ | GPHDTSSGGVRPNLHHTSKKEKRENR | 33 |
| | KVPFYSHSVTSRGNV | |
| H | KHPTYRQ | 34 |
| I | HPMSAPR | 35 |
| J | MPKYYLQ | 36 |

¹There was a multiple DNA fragment insertion in these clones.

### Example 2

### Selection of High Affinity Hair-Binding Phage Peptides Using a Modified Method

The purpose of this Example was to identify hair-binding phage peptides with a higher binding affinity.

The hairs that were treated with the acidic elution buffer, as described in Example 1, were washed three more times with the elution buffer and then washed three times with TBST-0.5%. These hairs, which had acid resistant phage peptides still attached, were used to directly infect 500 μL of mid-log phase bacterial host cells, E. coli ER2738 (New England BioLabs), which were then grown in LB medium for 20 min and then mixed with 3 mL of agarose top (LB medium with 5 mM MgCl₂, and 0.7% agarose) at 45° C. This mixture was spread onto a LB medium/IPTG/S-GAL® plate (LB medium with 15 g/L agar, 0.05 g/L IPTG, and 0.04 g/L S-GAL®) and incubated overnight at 37° C. The black plaques were counted to calculate the phage titer. Single black plaques were randomly picked for DNA isolation and sequencing analysis, as described in Example 1. This process was performed on normal and bleached hair samples that were screened with the 7-mer and 12-mer phage display libraries, as described in Example 1. The amino acid sequences of these high affinity, hair-binding phage peptides are given in Tables 4-7.

TABLE 4

Amino Acid Sequences of High Affinity. Normal
Hair-Binding Phage Peptides from 7-Mer Library

| | | SEQ |
|---|---|---|
| Clone | | ID |
| ID | Amino Acid Sequence | NO: |
| D5 | GPHDTSSGGVRPNLHHTSKKEKRENRKVPFYSHSVTS | 33 |
| | RGNV¹ | |
| A36 | MHAHSIA | 37 |
| B41 | TAATTSP | 38 |

¹There was a multiple DNA fragment insertion in this clone.

TABLE 5

Amino Acid Sequences of High Affinity. Bleached
Hair-Binding Phage Peptides from 7-Mer Library

| | | SEQ |
|---|---|---|
| Clone | | ID |
| ID | Amino Acid Sequence | NO: |
| D39 | LGIPQNL | 39 |
| B1 | TAATTSP | 38 |

TABLE 6

Amino Acid Sequences of High Affinity. Normal
Hair-Binding Phage Peptides from 12-Mer Library

| | | |
|---|---|---|
| | | SEQ |
| Clone | | ID |
| ID | Amino Acid Sequence | NO: |

| | | |
|---|---|---|
| C2 | AKPISQHLQRGS | 40 |
| A3 | APPTPAAASATT | 41 |
| F9 | DPTEGARRTIMT | 42 |
| A19 | EQISGSLVAAPW | 43 |
| F4 | LDTSFPPVPFHA | 44 |
| F35 | LPRIANTWSPS | 45 |
| D21 | RTNAADHPAAVT | 46 |
| C10 | SLNWVTIPGPKI | 47 |
| C5 | TDMQAPTKSYSN | 48 |
| D20 | TIMTKSPSLSCG | 49 |
| C18 | TPALDGLRQPLR | 50 |
| A20 | TYPASRLPLLAP | 51 |
| C13 | AKTHKHPAPSYS | 52 |
| G-D20 | YPSFSPTYRPAF | 53 |
| A23 | TDPTPFSISPER | 54 |
| F67 | SQNWQDSTSYSN | 55 |
| F91 | WHDKPQNSSKST | 56 |
| G-F1 | LDVESYKGTSMP | 4 |

TABLE 7

Amino Acid Sequences of High Affinity, Bleached
Hair-Binding Phage Peptides from 12-Mer Library

| | | SEQ |
|---|---|---|
| Clone | | ID |
| ID | Amino Acid Sequence | NO: |
| A5 | EQISGSLVAAPW | 43 |
| C4 | NEVPARNAPWLV | 57 |
| D30 | NSPGYQADSVAIG | 58 |
| C44 | AKPISQHLQRGS | 40 |
| E66 | LDTSFPPVPFHA | 44 |
| C45 | SLNWVTIPGPKI | 47 |
| E18 | TQDSAQKSPSPL | 59 |

### Example 3

### Selection of High Affinity Fingernail-Binding Phage Peptides

The purpose of this Example was to identify phage peptides that have a high binding affinity to fingernails. The modified biopanning method described in Example 2 was used to identify high affinity, fingernail-binding phage-peptide clones.

Human fingernails were collected from test subjects. The fingernails were cleaned by brushing with soap solution, rinsed with deionized water, and allowed to air-dry at room temperature. The fingernails were then powdered under liquid N₂, and 10 mg of the fingernails was added to each well of a 96-well filter plate. The fingernail samples were treated for 1 h with blocking buffer consisting of 1 mg/mL BSA in TBST-0.5%, and then washed with TBST-0.5%. The fingernail samples were incubated with phage library (Ph.D-12 Phage Display Peptide Library Kit), and washed 10 times using the same conditions described in Example 1. After the acidic elution step, described in Example 1, the fingernail samples were washed three more times with the elution buffer and then washed three times with TBST-0.5%. The acid-treated fingernails, which had acid resistant phage peptides still attached, were used to directly infect E. coli ER2738 cells as described in Example 2. This biopanning process was repeated three times. A total of 75 single black phage plaques were picked randomly for DNA isolation and sequencing analysis and two repeated clones were identified. The amino acid sequences of these phage peptides are listed in Table 8. These fingernail binding peptides were also found to bind well to bleached hair.

TABLE 8

Amino Acid Sequences of High Affinity Finger-
nail-Binding Phage Peptides

| | | SEQ | |
|---|---|---|---|
| Clone | Amino Acid | ID | |
| ID | Sequence | NO: | Frequency¹ |
| F01 | ALPRIANTWSPS | 60 | 15 |
| D05 | YPSFSPTYRPAF | 53 | 26 |

¹The frequency represents the number of identical sequences that occurred out of 75 sequenced clones.

### Example 4

### Selection of High Affinity Skin-Binding Phage Peptides

The purpose of this Example was to identify phage peptides that have a high binding affinity to skin. The modified biopanning method described in Examples 2 and 4 was used to identify the high affinity, skin-binding phage-peptide clones. Pig skin served as a model for human skin in the process.

The pig skin was prepared as described in Example 1. Three rounds of screening were performed with the custom, pig skin bottom biopanning apparatus using the same procedure described in Example 4. A total of 28 single black phage plaques were picked randomly for DNA isolation and sequencing analysis and one repeated clone was identified. The amino acid sequence of this phage peptide, which appeared 9 times out of the 28 sequences, was TPFHSPENAPGS, (SK-1) given as SEQ ID NO:61.

### Example 5

### Quantitative Characterization of the Binding Affinity of Hair-Binding Phage Clones

The purpose of this Example was to quantify the binding affinity of phage clones by titering and ELISA.

Titering of Hair-Binding Phage Clones:

Phage clones displaying specific peptides were used for comparing the binding characteristics of different peptide sequences. A titer-based assay was used to quantify the phage binding. This assay measures the output pfu retained by 10 mg of hair surfaces, having a signal to noise ratio of 10³ to 10⁴. The input for all the phage clones was 10¹⁴ pfu. It should be emphasized that this assay measures the peptide-expressing phage particle, rather than peptide binding.

Normal hairs were cut into 0.5 cm lengths and 10 mg of the cut hair was placed in each well of a 96-well filter plate (Qiagen). Then, the wells were filled with blocking buffer containing 1 mg/mL BSA in TBST-0.5% and incubated for 1 h at 4° C. The hairs were washed 5 times with TBST-0.5%. The wells were then filled with 1 mL of TBST-0.5% containing 1 mg/mL BSA and then purified phage clones (10¹⁴ pfu) were added to each well. The hair samples were incubated for 15 min at room temperature and then washed 10 times with TBST-0.5%. The hairs were transferred to a clean well and 1.0 mL of a non-specific elution buffer, consisting of 1 mg/mL BSA in 0.2 M Glycine-HCl at pH 2.2, was added to each well. The samples were incubated for 10 min and then 160 μL of neutralization buffer (1 M Tris-HCl, pH 9.2) was added to each well. The eluted phage from each well were transferred to a new tube for titering and sequencing analysis.

To titer the bound phage, the eluted phage was diluted with SM buffer to prepare 10-fold serial dilutions of 10¹ to 10⁸. A 10 μL aliquot of each dilution was incubated with 200 μL of mid-log phase E. coli ER2738 (New England BioLabs), and grown in LB medium for 20 min and then mixed with 3 mL of agarose top (LB medium with 5 mM MgCl₂, and 0.7% agarose) at 45° C. This mixture was spread onto a LB medium/IPTG/Xgal (5-bromo-4-chloro-3-indolyl-β-D-galactopyranoside) plate (LB medium with 15 g/L agar, 0.05 g/L IPTG, and 0.04 g/L Xgal) and incubated overnight at 37° C. The blue plaques were counted to calculate the phage titers, which are given in Table 9.

TABLE 9
Titer of Hair-Binding Phage Clones

| Clone ID | SEQ ID NO: | Phage Titer |
|---|---|---|
| A | 28 | 7.50 × 10⁴ |
| B | 29 | 1.21 × 10⁵ |
| D | 30 | 8.20 × 10⁴ |
| E | 31 | 1.70 × 10⁵ |
| F | 32 | 1.11 × 10⁶ |
| G | 33 | 1.67 × 10⁸ |
| H | 34 | 1.30 × 10⁶ |
| 1 | 35 | 1.17 × 10⁶ |
| J | 36 | 1.24 × 10⁶ |

Characterization of Hair-Binding Phage Clones by ELISA:

Enzyme-linked immunosorbent assay (ELISA) was used to evaluate the hair-binding specificity of selected phage-peptide clones. Phage-peptide clones identified in Examples 1 and 2 along with a randomly chosen control G-F9, KHGPDLLRSAPR (given as SEQ ID NO:63) were amplified. More than 1014 pfu (plaque forming units) phage were added to pre-blocked hair surfaces. The same amount of phage was also added to pre-blocked pig skin surfaces as a control to demonstrate the hair-binding specificity.

A unique hair or pig skin-bottom 96-well apparatus was created by applying one layer of PARAFILM® under the top 96-well block of a Minifold I Dot-Blot System (Schleicher & Schuell, Inc., Keene, N.H.), adding hair or a layer of hairless pig skin on top of the PARAFILM® cover, and then tightening the apparatus. For each clone to be tested, the hair-covered well was incubated for 1 h at room temperature with 200 μL of blocking buffer, consisting of 2% non-fat dry milk (Schleicher & Schuell, Inc.) in TBS. A second Minifold system with pig skin at the bottom of the wells was treated with blocking buffer simultaneously to serve as a control. The blocking buffer was removed by inverting the systems and blotting them dry with paper towels. The systems were rinsed 6 times with wash buffer consisting of TBST-0.05%. The wells were filled with 200 μL of TBST-0.5% containing 1 mg/mL BSA and then 10 μL (over 1012 copies) of purified phage stock was added to each well. The samples were incubated at 37° C. for 15 min with slow shaking. The non-binding phage was removed by washing the wells 10 to 20 times with TBST-0.5%. Then, 100 μL of horseradish peroxidase/anti-M13 antibody conjugate (Amersham USA, Piscataway, N.J.), diluted 1:500 in the blocking buffer, was added to each well and incubated for 1 h at room temperature. The conjugate solution was removed and the wells were washed 6 times with TBST-0.05%. TMB (3,3′,5,5′-tetramethylbenzidine) substrate (200 μL), obtained from Pierce Biotechnology (Rockford, Ill.) was added to each well and the color was allowed to develop for between 5 to 30 min, typically for 10 min, at room temperature. Then, stop solution (200 μL of 2 M H₂SO₄) was added to each well and the solution was transferred to a 96-well plate and the A₄₅₀ was measured using a microplate spectrophotometer (Molecular Devices, Sunnyvale, Calif.). The resulting absorbance values, reported as the mean of at least three replicates, and the standard error of the mean (SEM) are given in Table 10.

TABLE 10
Results of ELISA Assay with Skin and Hair

| | SEQ ID | Hair | | Pig Skin | |
|---|---|---|---|---|---|
| Clone ID | NO: | A₄₅₀ | SEM | A₄₅₀ | SEM |
| G-F9 | 63 | 0.074 | 0.057 | −0.137 | 0.015 |
| (Control) | | | | | |
| D21 | 46 | 1.051 | 0.16 | 0.04 | 0.021 |
| D39 | 39 | 0.685 | 0.136 | 0.086 | 0.019 |
| D5 | 33 | 0.652 | 0.222 | 0.104 | 0.023 |
| A36 | 37 | 0.585 | 0.222 | 0.173 | 0.029 |
| C5 | 48 | 0.548 | 0.263 | 0.047 | 0.037 |
| C10 | 47 | 0.542 | 0.105 | 0.032 | 0.012 |
| A5 | 43 | 0.431 | 0.107 | 0.256 | 0.022 |
| B1 | 38 | 0.42 | 0.152 | 0.127 | 0.023 |
| D30 | 58 | 0.414 | 0.119 | 0.287 | 0.045 |
| C13 | 52 | 0.375 | 0.117 | 0.024 | 0.016 |
| C18 | 50 | 0.34 | 0.197 | 0.132 | 0.023 |

As can be seen from the data in Table 10, all the hair-binding clones had a significantly higher binding affinity for hair than the control. Moreover, the hair-binding clones exhibited various degrees of selectivity for hair compared to pig skin. Clone D21 had the highest selectivity for hair, having a very strong affinity for hair and a very low affinity for pig skin.

### Example 6

### Confirmation of Peptide Binding Specificity and Affinity

The purpose of this Example was to test the peptide binding site specificity and affinity of the hair-binding peptide D21 using a competition ELISA. The ELISA assay only detects phage particles that remain bound to the hair surface. Therefore, if the synthetic peptide competes with the phage particle for the same binding site on hair surface, the addition of the synthetic peptide into the ELISA system will significantly reduce the ELISA results due to the peptide competition.

The synthetic hair-binding peptide D21, given as SEQ ID NO:46 was synthesized by SynPep (Dublin, Calif.). As a control, an unrelated synthetic skin-binding peptide (SK-1), given as SEQ ID NO:61, was added to the system. The experimental conditions were similar to those used in the ELISA method described in Example 5. Briefly, 100 μL of Binding Buffer (1×TBS with 0.1% Tween® 20 and 1 mg/mL BSA) and 10¹¹ pfu of the pure D21 phage particles were added to each well of the 96-well filter plate, which contained a sample of normal hair. The synthetic peptide (100 μg) was added to each well (corresponding to concentration of 0.8 mM). The reactions were carried out at room temperature for 1 h with gentle shaking, followed by five washes with TBST-0.5%. The remaining steps were identical to the those used in the ELISA method described in Example 5. The ELISA results, presented as the absorbance at 450 nm (A₄₅₀), are shown in Table 11. Each individual ELISA test was performed in triplicate; the values in Table 11 are the means of the triplicate determinations.

TABLE 11
Results of Peptide Competition ELISA

| Sample | A₄₅₀ | SEM |
|---|---|---|
| Antibody-Conjugate | 0.199 | 0.031 |
| Phage D21 | 1.878 | 0.104 |
| Phage D21 and D21 | 1.022 | 0.204 |
| Peptide | | |
| Phage D21 and | 2.141 | 0.083 |
| Control Peptide | | |

These results demonstrated that the synthetic peptide D21 does compete with the phage clone D21 for the same binding sites on the hair surface.

### Example 7

### Selection of Shampoo-Resistant Hair-Binding Phage-Peptides Using Biopanning

The purpose of this Example was to select shampoo-resistant hair-binding phage-peptides using biopanning with shampoo washes.

In order to select shampoo-resistant hair-binding peptides, a biopanning experiment using 12-mer phage peptide libraries against normal and bleached hairs was performed, as described in Example 2. Instead of using normal TBST buffer to wash-off the unbounded phage, the phage-complexed hairs were washed with 10%, 30% and 50% shampoo solutions (Pantene Pro-V shampoo, Sheer Volume, Proctor & Gamble, Cincinnati, Ohio), for 5 min in separate tubes, followed by six TBS buffer washes. The washed hairs were directly used to infect host bacterial cells as described in the modified biopanning method, described in Example 2.

A potential problem with this method is the effect of the shampoo on the phage's ability to infect bacterial host cells. In a control experiment, a known amount of phage particles was added to a 10% shampoo solution for 5 min, and then a portion of the solution was used to infect bacterial cells. The titer of the shampoo-treated phage was 90% lower than that of the untreated phage. The 30% and 50% shampoo treatments gave even more severe damage to the phage's ability to infect host cells. Nevertheless, two shampoo-resistant hair-binding phage-peptides were identified, as shown in Table 12.

TABLE 12

Peptide Sequences of Shampoo-Resistant Hair-
binding Phage Peptides Identified Using the
Biopanning Method

| | | | SEQ |
|---|---|---|---|
| | | | ID |
| Clone | Sequence | Target | NO: |
| I-B5 | TPPELLHGDPRS | Normal and | 66 |
| | | Bleached Hair | |
| H-B1 | TPPTNVLMLATK | Normal Hair | 69 |

### Example 8

### Selection of Shampoo-Resistant Hair-Binding Phage-Peptides Using PCR

The purpose of this Example was to select shampoo-resistant hair-binding phage-peptides using a PCR method to avoid the problem of shampoo induced damage to the phage. This principle of the PCR method is that DNA fragments inside the phage particle can be recovered using PCR, regardless of the phage's viability, and that the recovered DNA fragments, corresponding to the hair-binding peptide sequences, can then been cloned back into a phage vector and packaged into healthy phage particles.

Biopanning experiments were performed using 7-mer and 12-mer phage-peptide libraries against normal and bleached hairs, as described in Example 1. After the final wash, the phage-treated hairs were subjected to 5 min of shampoo washes, followed by six TBS buffer washes. The shampoo-washed hairs were put into a new tube filled with 1 mL of water, and boiled for 15 min to release the DNA. This DNA-containing, boiled solution was used as a DNA template for PCR reactions. The primers used in the PCR reaction were primers: M13KE-1412 Forward 5′-CAAGCCTCAGCGACCGAATA-3′, given as SEQ ID NO:67 and M13KE-1794 Reverse 5′-CGTAACACTGAGTTTCGTCACCA-3′, given SEQ ID NO:68. The PCR conditions were: 3 min denaturing at 96° C., followed by 35 cycles of 94° C. for 30 sec, 50° C. for 30 sec and 60° C. for 2 min. The PCR products (˜400 bp), and M13KE vector (New England BioLabs) were digested with restriction enzymes Eag I and Acc65 I. The ligation and transformation conditions, as described in the Ph.D.™ Peptide Display Cloning System (New England Biolabs), were used. The amino acid sequence of the resulting shampoo-resistant hair-binding phage-peptide is NTSQLST, (KF-11) given as SEQ ID NO:70.

### Example 9

### Determination of the Affinity of Hair-Binding and Skin-Binding Peptides

The purpose of this Example was to determine the affinity of the hair-binding and skin-binding peptides for their respective substrates, measured as MB₅₀ values, using an ELISA assay.

Hair-binding and skin-binding peptides were synthesized by SynPep Inc. (Dublin, Calif.). The peptides were biotinylated by adding a biotinylated lysine residue at the C-terminus of the amino acid binding sequences for detection purposes and an amidated cysteine was added to the C-terminus of the sequence. The amino acid sequences of the peptides tested are given as SEQ ID NOs:71-74 as shown in Table 13.

For hair samples, the procedure used was as follows. The setup of the surface specific 96-well system used was the same as that described in Example 5. Briefly, the 96-wells with hair or pig skin surfaces were blocked with blocking buffer (SUPERBLOCK™ from Pierce Chemical Co., Rockford, Ill.) at room temperature for 1 h, followed by six washes with TBST-0.5%, 2 min each, at room temperature. Various concentrations of biotinylated, binding peptide were added to each well, incubated for 15 min at 37° C., and washed six times with TBST-0.5%, 2 min each, at room temperature. Then, streptavidin-horseradish peroxidase (HRP) conjugate (Pierce Chemical Co.) was added to each well (1.0 μg per well), and incubated for 1 h at room temperature. After the incubation, the wells were washed six times with TBST-0.5%, 2 min each at room temperature. Finally, the color development and the measurement were performed as described in Example 5.

For the measurement of MB₅₀ of the peptide-skin complexes, the following procedure was used. First, the pigskin was treated to block the endogenous biotin in the skin. This was done by adding streptavidin to the blocking buffer. After blocking the pigskin sample, the skin was treated with D-biotin to block the excess streptavidin binding sites. The remaining steps were identical to those used for the hair samples.

The results were plotted as A₄₅₀ versus the concentration of peptide using GraphPad Prism 4.0 (GraphPad Software, Inc., San Diego, Calif.). The MB₅₀ values were calculated from Scatchard plots and are summarized in Table 13. The results demonstrate that the binding affinity of the hair-binding peptides (D21, SEQ ID NO: 46; F35, SEQ ID NO: 45; and I-B5, SEQ ID NO: 66) and the skin-binding peptide SK-1 (SEQ ID NO: 61) for their respective substrate was high, while the binding affinity of the hair-binding peptides (D-21 and I-B5) for skin was relatively low.

TABLE 13
Summary of MB₅₀ Values for Hair and Skin-Binding Peptides

| Binding | Peptide Sequence | | |
|---|---|---|---|
| Peptide | Tested* | Substrate | MB₅₀, M |
| D21 | SEQ ID NO: 71 | Normal Hair | 2 × 10⁻⁶ |
| F35 | SEQ ID NO: 72 | Bleached Hair | 3 × 10⁻⁶ |
| I-B5 | SEQ ID NO: 73 | Normal and | 3 × 10⁻⁷ |
| | | Bleached Hair | |
| D21 | SEQ ID NO: 71 | Pig Skin | 4 × 10⁻⁵ |
| I-B5 | SEQ ID NO: 73 | Pig Skin | >1 × 10⁻⁴ |
| SK-1 | SEQ ID NO: 74 | Pig Skin | 7 × 10⁻⁷ |

*The peptides tested were biotinylated by the addition of a biotinylated lysine residue at the C-terminus of the amino acid binding sequences and an amidated cysteine was added to the C-terminus of the sequence following the biotinylated lysine residue.

### Example 10

### Conditioning Reagents Made Recombinantly Comprising Peptide Linkers

The purpose of this Example was to demonstrate the synthesis of peptide-based conditioning reagents comprising combinations of peptide spacers, hair-binding peptides and conditioning peptides derived from silk-like proteins and keratin.

The sequences of the peptide-based body surface conditioning reagents prepared in these Examples are given in Table 14.

TABLE 14

| | | SEQ |
|---|---|---|
| | | ID |
| Conjugate | Peptide Sequence | NO: |
| HC77648 | TPPELLHGEPRS (Hair binder)-GGP | 161 |
| | (Spacer)-TPPELLHGEPRS (Hair binder)- | |
| | GPGVG (Spacer)-GAGAGYGAGAGYGAGAGYGAGA | |
| | GY (Semicrystalline Silkx4) | |
| HC77649 | NTSQLST (Hair binder)-GGP (Spacer)- | 163 |
| | NTSQLST (Hair binder)-GPGVG (Spacer)- | |
| | AEQFRNQAEQFRNQAEQFRNQAEQFRNQ | |
| | (Keratinx4) | |
| HC77651 | TPPELLHGEPRS (Hair binder)-GGP | 166 |
| | (Spacer)-TPPELLHGEPRS (Hair binder)- | |
| | GPGVG (Spacer)-GAGAGYGAGAGYGAGAGYGAGA | |
| | GY (Semicrystalline Silkx4)-TPPELLHGE | |
| | PRS(Hair binder)-GGP (Spacer)-TPPELLH | |
| | GEPRS (Hair binder)- | |

DNA sequences were designed to encode these peptides using favorable codons for E. coli and avoiding sequence repeats and mRNA secondary structure. The gene DNA sequence was designed by DNA 2.0 Inc, Menlo Park, Calif., using commercially available software described in Gustafsson, C. et al. Trends in Biotechnol. (2004) 22(7):346-355. In each case the sequence encoding the amino acid sequence was followed by two termination codons and a recognition site for endonuclease AscI. The GS amino acid sequence at the N-terminus was encoded by a recognition site for endonuclease BamHI (GGA/TCC). The DNA sequences used are given in Table 15.

TABLE 15

| | | SEQ |
|---|---|---|
| | | ID |
| Conjugate | Nucleic acid Sequence | NO: |
| HC77648 | GGATCCGACCCTGGCACCCCTCCAGAACTGCTGCACG | 167 |
| | GCGAACCACGCTCTGGTGGCCCGACGCCTCCAGAACT | |
| | GCTGCATGGCGAACCGCGCTCCGGTCCGGGTGTGGGC | |
| | GGTGCTGGTGCGGGCTATGGTGCGGGTGCAGGCTATG | |
| | GCGCTGGCGCTGGCTACGGTGCGGGCGCAGGCTACTG | |
| | ATAAGGCGCGCC | |
| HC77649 | GGATCCGACCCTGGTAATACTTCTCAACTGTCTACTG | 168 |
| | GTGGTCCTAATACTAGCCTGCAGTCTACGGGCCCAGG | |
| | TGTAGGTGCTGAACAATTCCGCAACCAGGCGGAACAG | |
| | TTTCGTAACCAGGCTGAGCAGTTCCGTAACCAAGCTG | |
| | AACAGTTCCGTAATCAATAATAAGGCGCGCC | |
| HC77651 | GGATCCGACCCTGGCACTCCTCCTGAACTGCTGCACG | 169 |
| | GTGAACCACGCTCCGGTGGCCCGACTCCGCCGGAGCT | |
| | GCTGCACGGTGAACCGCGTTCTGGCCCAGGTGTGGGT | |
| | GGCGCCGGTGCTGGTTATGGTGCCGGTGCGGGCTACG | |
| | GTGCTGGTGCTGGCTACGGTGCGGGCGCAGGCTACAC | |
| | TCCGCCTGAGCTGCTGCATGGCGAACCACGTTCTGGC | |
| | GGTCCGACGCCTCCAGAACTGCTGCATGGTGAGCCGC | |
| | GTTCCTGATGAGGCGCGCC | |

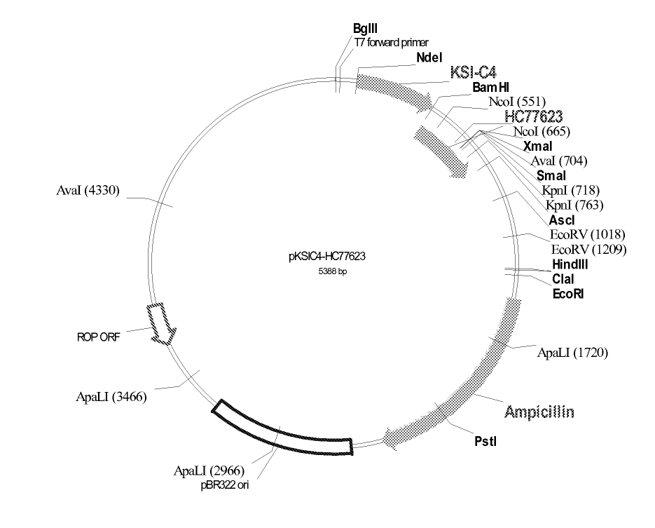

Genes were assembled from synthetic oligonucleotides and cloned in a standard plasmid cloning vector by DNA 2.0. Sequences were verified by DNA sequencing by DNA 2.0. The synthetic genes were excised from the cloning vector with endonucleases BamHI and AscI and ligated into an expression vector using standard recombinant DNA methods. The vector pKSIC4-HC77623 (FIG. 1) was derived from the commercially available vector pDEST17 (Invitrogen, Carlsbad, Calif.). It includes sequences derived from the commercially available vector pET31 b (Novagen, Madison, Wis.) that encode a fragment of the enzyme ketosteroid isomerase (KSI). The KSI fragment was included as a fusion partner to promote partition of the peptides into insoluble inclusion bodies in E. coli. The KSI-encoding sequence from pET31 b was modified using standard mutagenesis procedures (QuickChange II, Stratagene, La Jolla, Calif.) to include three additional Cys codons, in addition to the one Cys codon found in the wild type KSI sequence. The plasmid pKSIC4-HC77623 was constructed using standard recombinant DNA methods well known to those skilled in the art (FIG. 1). Its complete DNA sequence is given in SEQ ID NO: 172.

DNA sequences encoding HC77648, HC77649, and HC77651 were inserted into pKSIC4-HC77623 by substituting for sequences in the vector between the BamHI and AscI sites. Plasmid DNA containing the peptide encoding sequences and vector DNA were digested with endonucleases BamHI and AscI, then the peptide-encoding sequences and vector DNA were mixed and ligated by phage T4 DNA ligase using standard DNA cloning procedures well known to those skilled in the art. Correct constructs, in which the sequences encoding HC77648, HC77649, and HC77651 were respectively inserted into pKSIC4-HC77623 were identified by restriction analysis and verified by DNA sequencing, again using standard methods.

In these constructs, the sequences encoding the peptides of interest were substituted for those encoding HC77623. They became operably linked to the bacteriophage T7 gene 10 promoter and expressed as a fusion protein, fused with the variant KSI partner. The expression plasmids are designated pKSIC4-HC77648, pKSIC4-HC77649, and pKSIC4-HC77651.

To test the expression of the proteins, the expression plasmids were transformed into the BL21-AI E. coli strain (Invitrogen catalog no. C6070-03). To produce the recombinant protein, 50 mL of LB-ampicillin broth (10 g/L bacto-tryptone, 5 g/L bacto-yeast extract, 10 g/L NaCl, 100 mg/L ampicillin, pH 7.0) was inoculated with one colony of the transformed bacteria and the culture was shaken at 37° C. until the OD₆₀₀ reached 0.6. The expression was induced by adding 0.5 mL of 20% L-arabinose to the culture and shaking was continued for another 4 h. Analysis of the cell protein by polyacrylamide gel electrophoresis demonstrated the production of the peptide conjugates.

What is claimed is:

1. A peptide based conditioning reagent having the general structure [[(BSBP)ₘ-S_{q}]ₓ-[(CP)ₙ-Sᵣ]_{z}]y, wherein
a) BSBP is a body surface-binding peptide;
b) CP is a conditioning peptide;
c) S is a molecular spacer; and
d) m, n, x and z independently range from 1 to about 10, y is from 1 to about 5, and where q and r are each independently 0 or 1, and wherein the peptide based conditioning reagent has a molecular weight of less than about 200,000 Daltons.

2. A conditioning reagent according to claim 1 wherein the body surface-binding peptide is selected from the group consisting of a hair-binding peptide, a skin-binding peptide, and a nail binding peptide.

3. A peptide-based conditioning reagent according to claim 1 wherein the body surface-binding peptide is from about 7 to about 50 amino acids in length and has a binding affinity for a body surface, measured as MB₅₀, equal to or less than 10⁻⁵ M.

4. A peptide-based conditioning reagent according to claim 2 wherein the hair-binding peptide is selected from the group consisting of SEQ ID NOs: 38, 39, 40, 43, 47, 57, 58, 59, and 66.

5. A peptide-based conditioning reagent according to claim 2 wherein the skin-binding peptide has the amino acid sequence as set forth in SEQ ID NO: 61.

6. A peptide-based conditioning reagent according to claim 2 wherein the nail-binding peptide is selected from the group consisting of SEQ ID NOs: 53 and 60.

7. A peptide-based conditioning reagent according to claim 1 wherein the molecular spacer is selected from the group consisting of ethanolamine, ethylene glycol, polyethylene with a chain length of 6 carbon atoms, polyethylene glycol with 3 to 6 repeating units, phenoxyethanol, propanolamide, butylene glycol, butyleneglycolamide, propyl phenyl chains, ethyl alkyl chains, propyl alkyl chains, hexyl alkyl chains, steryl alkyl chains, cetyl alkyl chains, and palmitoyl alkyl chains.

8. A peptide-based conditioning reagent according to claim 1 wherein the molecular spacer is a peptide comprising from 2 to about 50 amino acids.

9. A peptide-based conditioning reagent according to claim 8 wherein the molecular spacer comprises peptide sequences selected from the group consisting of SEQ ID NOs: 123 and 124.

10. A peptide-based conditioning reagent according to claim 1 wherein the conditioning peptide (CP) comprises a repeat sequence protein selected from the group consisting of, silk, keratin, abductin, elastin, byssus, flagelliform silk-like protein, gluten high molecular weight (HMW) subunit, titin, fibronectin, laminin, collagen, gliadin, glue polypolypeptide, ice nucleating protein, keratin, mucin and resilin.

11. A peptide-based conditioning reagent according to claim 10 wherein the conditioning peptide comprises a peptide repeat sequence selected from the group consisting of SEQ ID NO: 143; SEQ ID NO: 144; SEQ ID NO: 145; SEQ ID NO: 126; SEQ ID NO: 118; SEQ ID NO: 127; SEQ ID NO: 128; SEQ ID NO: 128; SEQ ID NO: 130; SEQ ID NO: 170; SEQ ID NO: 131; SEQ ID NO: 132; SEQ ID NO: 133; SEQ ID NO: 135; SEQ ID NO: 136; SEQ ID NO: 137; SEQ ID NO: 138; SEQ ID NO: 139; SEQ ID NO: 140; SEQ ID NO: 158 and SEQ ID NO: 141.

12. A peptide-based conditioning reagent according to claim 10 wherein the silk-like protein has the general formula:
[(A)ₑ-(E)_{f}-(S)_{f}—(X)ₚ-(E)_{f}-(S)_{f]}ᵢ
wherein:
A or E are different non-crystalline soft segments of about 10 to 25 amino acids having at least 55% Gly;
S is a semi-crystalline segment of about 6 to 12 amino acids having at least 33% Ala, and 50% Gly;
X is a crystalline hard segment of about 6 to 12 amino acids having at least 33% Ala, and 50% Gly; and
wherein,
e is 2, 4, 8, 16, 32, 64, or 128;
each f is independently 0, 1, 2, 4, 8, 16, 32, 64, or 128;
p is 2, 4, 8, 16, 32, 64, or 128;
i is 1 to 128; and
where p is a number greater than n or f.

13. A peptide-based conditioning reagent according to claim 12 wherein the silk-like protein is defined by a formula selected from the group consisting of: [(A)₄-(X)₈]₈, [(A)₄-(X)₈-(S)]₈, [(A)₄-(X)₈-(E)]₈, [(A)₈-(X)₈]₈, [(A)₄-(S)-(X)₈]₈, [(A)₄-(S)₂-(X)₈]₈, [(A)₄-(E)-(X)₈-(E)]₈, [(A)₄-(E)-(X)₈]₈, [(A)₄-(S)-(X)₈-(E)]8, and [(A)₄-(S)₂-(X)₈-(E)]₈.

14. A peptide-based conditioning reagent according to claim 13 wherein
A has an amino acid sequence consisting of SEQ ID NO: 143;
E has an amino acid sequence consisting of SEQ ID NO: 144;
S has an amino acid sequence consisting of SEQ ID NO: 145; and
X has an amino acid sequence consisting of SEQ ID NO: 126.

15. A peptide-based conditioning reagent according to claim 10 wherein the silk-like protein is a spider silk variant having the general formula:
(SEQ ID NO:150-157)
[ACGQGGYGGLGXQGAGRGGLGGQGAGA_{g}GG]ₕ
wherein X is S, G or N; g=0-7 and h=1-75, and wherein the value of g determines the number of repeats in the variant protein and wherein the formula encompasses variations selected from the group consisting of:
(a) when g is 0 the sequence encompassing
AGRGGLGGQGAGA_{g}GG (SEQ ID NO:147) is deleted;
(b) deletions other than the poly-alanine sequence, limited by the value of g will encompass integral multiples of three consecutive residues;
(c) the deletion of GYG in any repeat is accompanied by deletion of GRG in the same repeat; and
(d) where a first repeat where n=0 is deleted, the first repeat is preceded by a second repeat where n=6; and
wherein the full-length protein is encoded by a gene or genes and wherein said gene or genes are not endogenous to the Nephila clavipes genome.

16. A peptide-based conditioning reagent according to claim 1 wherein:
a) BSBP has an amino acid sequence selected from the group consisting of SEQ ID NOs: 4, 38, 39, 40, 43, 44, 47, and 53, 54, 55, and 56.
b) S has an amino acid sequence selected from the group consisting of SEQ ID NO: 123 and 124;
c) CP has an amino acid sequence comprising at least one repeat sequence selected from the group consisting of SEQ ID NO: 143; SEQ ID NO: 144; SEQ ID NO: 145; SEQ ID NO: 126; SEQ ID NO: 118; SEQ ID NO: 127; SEQ ID NO: 128; SEQ ID NO: 128; SEQ ID NO: 130; SEQ ID NO: 170; SEQ ID NO: 131; SEQ ID NO: 132; SEQ ID NO: 133; SEQ ID NO: 135; SEQ ID NO: 136; SEQ ID NO: 137; SEQ ID NO: 138; SEQ ID NO: 139; SEQ ID NO: 140; SEQ ID NO: 158 and SEQ ID NO: 141.

17. A peptide-based conditioning regent according to claim 1 comprising a peptide conjugate having an amino acid sequence selected from the group consisting of SEQ ID NOs: 161, 163, and 166.

18. A peptide-based conditioning reagent according to claim 1 wherein the conditioning reagent is from about 14 to about 200 amino acids in length.

19. A peptide-based conditioning reagent according to claim 1 wherein the body surface-binding peptide is isolated by a process comprising the steps of:
(i) providing a library of combinatorially generated phage-peptides;
(ii) contacting the library of (i) with a body surface to form a reaction solution comprising:
(A) phage-peptide-body surface complex;
(B) unbound body surface, and
(C) uncomplexed peptides;
(iii) isolating the phage-peptide-body surface complex of (ii);
(iv) eluting the weakly bound peptides from the isolated peptide complex of (iii);
(v) identifying the remaining bound phage-peptides either by using the polymerase chain reaction directly with the phage-peptide-body surface complex remaining after step (iv), or by infecting bacterial host cells directly with the phage-peptide-body surface complex remaining after step (iv), growing the infected cells in a suitable growth medium, and isolating and identifying the phage-peptides from the grown cells.

20. A peptide-based conditioning reagent according to claim 19 wherein the body surface is selected from the group consisting of hair, nails, and skin.

21. A personal care composition comprising an effective amount of the peptide-based conditioning reagent of claim 1, comprising a body surface-binding peptide and a conditioning peptide.

22. A personal care composition according to claim 21 wherein;
a) the body surface-binding peptide has affinity for a body surface selected from the group consisting of hair, nails, and skin; and
b) the body surface-binding peptide is from about 7 to about 50 amino acids in length and has a binding affinity for a body surface, measured as MB₅₀, equal to or less than 10⁻⁵ M.

23. A method for conditioning a body surface comprising applying a personal care composition comprising an effective amount of the peptide-based conditioning reagent of claim 1, comprising a body surface-binding peptide and a conditioning peptide, to a body surface under conditions wherein the body surface is conditioned.

24. A method according to claim 23 wherein the body surface is selected from the group consisting of hair, skin and nails.
